(19) United States
(12) Patent Application Publication
(10) Pub. No.: US 2006/0276534 A1
(43) Pub. Date: Dec. 7, 2006

(54) Methods of decreasing calcification

(75) Inventors: David Martin, Camarillo, CA (US); Juan Mariano Rodriguez Portillo, Cordoba (ES)

Correspondence Address: AMGEN INC., 1120 VETERANS BOULEVARD, SOUTH SAN FRANCISCO, CA, 94080, US

(73) Assignee: Amgen Inc., Thousand Oaks, CA (US)

(21) Appl. No.: 11/378,879

(22) Filed: Mar. 17, 2006

Related U.S. Application Data
(60) Provisional application No. 60/663,270, filed on Mar. 17, 2005.

(51) Int. Cl. A61K 31/277; A61K 31/36; A61K 31/195; A61K 31/18; A61K 31/165; A61K 31/137

(52) U.S. Cl. 514/464; 514/649; 514/621; 514/521; 514/602; 514/567; 514/597

(57) ABSTRACT

The present invention relates to methods of treating vascular calcification in subjects using calcimimetics.

### CROSS-REFERENCE TO RELATED APPLICATIONS

This application claims the benefit of United States Provisional Application No. 60/663,270 filed Mar. 17, 2005, which is incorporated herein by reference in its entirety.

### FIELD OF THE INVENTION

This invention relates generally to the field of medicine and, more specifically, to methods of decreasing, treating or preventing calcification.

### BACKGROUND OF THE INVENTION

Vascular calcification, a well-recognized and common complication of chronic kidney disease (CKD), increases the risk of cardiovascular morbidity and mortality (Giachelli, C. J Am Soc Nephrol 15: 2959-64, 2004; Raggi, P. et al. J Am Coll Cardiol 39: 695-701, 2002). While the causes of vascular calcification in CKD remain to be elucidated, associated risk factors include age, gender, hypertension, time on dialysis, diabetes and glucose intolerance, obesity, and cigarette smoking (Zoccali C. Nephrol Dial Transplant 15: 454-7, 2000). These conventional risk factors, however, do not adequately explain the high mortality rates from cardiovascular causes in the patient population. Recent observations suggest that certain abnormalities in calcium and phosphorus metabolism, resulting in a raised serum calcium-phosphorus product (Ca×P) contribute, among other factors, to the development of arterial calcification, and possibly to cardiovascular disease, in patients with end-stage renal disease (Goodman, W. et al. N Engl J Med 342: 1478-83, 2000; Guerin, A. et al. Nephrol Dial Transplant 15:1014-21, 2000; Vattikuti, R. & Towler, D. Am J Physiol Endocrinol Metab, 286: E686-96, 2004).

Another hallmark of advanced CKD is secondary hyperparathyroidism (HPT), characterized by elevated parathyroid hormone (PTH) levels and disordered mineral metabolism. The elevations in calcium, phosphorus, and Ca×P observed in patients with secondary HPT have been associated with an increased risk of vascular calcification (Chertow, G. et al. Kidney Int 62: 245-52, 2002; Goodman, W. et al. N Engl J Med 342: 1478-83, 2000; Raggi, P. et al. J Am Coll Cardiol 39: 695-701, 2002). Commonly used therapeutic interventions for secondary HPT, such as calcium-based phosphate binders and doses of active vitamin D sterols can result in hypercalcemia and hyperphosphatemia (Chertow, G. et al. Kidney Int 62: 245-52, 2002; Tan, A. et al. Kidney Int 51: 317-23, 1997; Gallieni, M. et al. Kidney Int 42: 1191-8, 1992), which are associated with the development or exacerbation of vascular calcification.

Vascular calcification is an important and potentially serious complication of chronic renal failure. Two distinct patterns of vascular calcification have been identified (Proudfoot, D & Shanahan, C. Herz 26: 245-51, 2001), and it is common for both types to be present in uremic patients (Chen, N. & Moe, S. Semin Nephrol 24: 61-8, 2004). The first, medial calcification, occurs in the media of the vessel in conjunction with a phenotypic transformation of smooth muscle cells into osteoblast-like cells, while the other, atherogenesis, is associated with lipid-laden macrophages and intimal hyperplasia.

Medial wall calcification can develop in relatively young persons with chronic renal failure, and it is common in patients with diabetes mellitus even in the absence of renal disease. The presence of calcium in the medial wall of arteries distinguishes this type of vascular calcification from that associated with atherosclerosis (Schinke T. & Karsenty G. Nephrol Dial Transplant 15: 1272-4, 2000). Atherosclerotic vascular calcification occurs in atheromatous plaques along the intimal layer of arteries (Farzaneh-Far A. JAMA 284: 1515-6, 2000). Calcification is usually greatest in large, well-developed lesions, and it increases with age (Wexler L. et al. Circulation 94: 1175-92, 1996; Rumberger J. et al. Mayo Clin Proc 1999, 74: 243-52.). The extent of arterial calcification in patients with atherosclerosis generally corresponds to severity of disease. Unlike medial wall calcification, atherosclerotic vascular lesions, whether or not they contain calcium, impinge upon the arterial lumen and compromise blood flow. The localized deposition of calcium within atherosclerotic plaques may happen because of inflammation due to oxidized lipids and other oxidative stresses and infiltration by monocytes and macrophages (Berliner J. et al. Circulation 91: 2488-96, 1995).

Some patients with end-stage renal disease develop a severe form of occlusive arterial disease called calciphylaxis or calcific uremic arteriolopathy. This syndrome is characterized by extensive calcium deposition in small arteries (Gipstein R. et al. Arch Intern Med 136: 1273-80, 1976; Richens G. et al. J Am Acad Dermatol. 6: 537-9, 1982). In patients with this disease, arterial calcification and vascular occlusion lead to tissue ischemia and necrosis. Involvement of peripheral vessels can cause ulceration of the skin of the lower legs or gangrene of the digits of the feet or hands. Ischemia and necrosis of the skin and subcutaneous adipose tissue of the abdominal wall, thighs and/or buttocks are features of a proximal form of calcific uremic arteriolopathy (Budisavljevic M. et al. J Am Soc Nephrol. 7: 978-82, 1996; Ruggian J. et al. Am J Kidney Dis 28: 409-14, 1996). This syndrome occurs more frequently in obese individuals, and women are affected more often than men for reasons that remain unclear (Goodman W. J. Nephrol. 15(6): S82-S85, 2002).

Current therapies to normalize serum mineral levels or to decrease, inhibit, or prevent calcification of vascular tissues or implants are of limited efficacy and cause unacceptable side effects. Therefore, there exists a need for an effective method of inhibiting and preventing vascular calcification.

### SUMMARY OF THE INVENTION

The present invention provides methods of inhibiting, decreasing, or preventing vascular calcification in a subject comprising administering a therapeutically effective amount of a calcimimetic compound to the subject. In one aspect, the vascular calcification can be atherosclerotic calcification. In another aspect, the vascular calcification can be medial calcification.

In one aspect, the subject can be suffering from chronic renal insufficiency or end-stage renal disease. In another aspect, the subject can be pre-dialysis. In a further aspect, the subject can be suffering from uremia. In another aspect, the subject can be suffering from diabetes mellitus I or II. In another subject, the subject can be suffering from a cardiovascular disorder. In one aspect, the subject can be human.

In one aspect, the calcimimetic compound can be a compound of the formula I 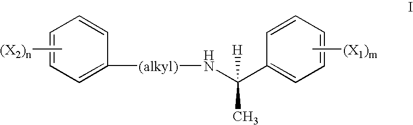
wherein:

X₁ and X₂, which may be identical or different, are each a radical chosen from CH₃, CH₃O, CH₃CH₂O, Br, Cl, F, CF₃, CHF₂, CH₂F, CF₃O, CH₃S, OH, CH₂OH, CONH₂, CN, NO₂, CH₃CH₂, propyl, isopropyl, butyl, isobutyl, t-butyl, acetoxy, and acetyl radicals, or two of X₁ may together form an entity chosen from fused cycloaliphatic rings, fused aromatic rings, and a methylene dioxy radical, or two of X₂ may together form an entity chosen from fused cycloaliphatic rings, fused aromatic rings, and a methylene dioxy radical; provided that X₂ is not a 3-t-butyl radical;

n ranges from 0 to 5;

m ranges from 1 to 5; and

the alkyl radical is chosen from C1-C3 alkyl radicals, which are optionally substituted with at least one group chosen from saturated and unsaturated, linear, branched, and cyclic C1-C9 alkyl groups, dihydroindolyl and thiodihydroindolyl groups, and 2-, 3-, and 4-piperidinyl groups;

or a pharmaceutically acceptable salt thereof.

In one aspect, the calcimimetic compound used in the methods of the invention can be N-(3-[2-chlorophenyl]-propyl)-R-α-methyl-3-methoxybenzylamine or a pharmaceutically acceptable salt thereof.

In another aspect, the calcimimetic compound can be a compound of the formula II

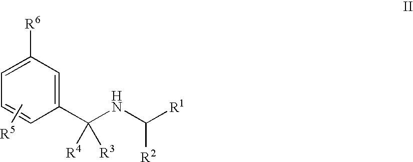

wherein:

R¹ is aryl, substituted aryl, heterocyclyl, substituted heterocyclyl, cycloalkyl, or substituted cycloalkyl;

R² is alkyl or haloalkyl;

R³ is H, alkyl, or haloalkyl;

R⁴ is H, alkyl, or haloalkyl;

each R⁵ present is independently selected from the group consisting of alkyl, substituted alkyl, alkoxy, substituted alkoxy, halogen, —C(═O)OH, —CN, —NR^{d}S(═O)ₘR^{d}, —NR^{d}C(═O)NR^{d}R^{d}, —NR^{d}S(═O)ₘNR^{d}R^{d}, or —NR^{d}C(═O)R^{d};

R⁶ is aryl, substituted aryl, heterocyclyl, substituted heterocyclyl, cycloalkyl, or substituted cycloalkyl;

each R^{a} is, independently, H, alkyl or haloalkyl;

each R^{b} is, independently, aryl, aralkyl, heterocyclyl, or heterocyclylalkyl, each of which may be unsubstituted or substituted by up to 3 substituents selected from the group consisting of alkyl, halogen, haloalkyl, alkoxy, cyano, and nitro;

each R^{c} is, independently, alkyl, haloalkyl, phenyl or benzyl, each of which may be substituted or unsubstituted;

each R^{d} is, independently, H, alkyl, aryl, aralkyl, heterocyclyl, or heterocyclylalkyl wherein the alkyl, aryl, aralkyl, heterocyclyl, and heterocyclylalkyl are substituted by 0, 1, 2, 3 or 4 substituents selected from alkyl, halogen, haloalkyl, alkoxy, cyano, nitro, R^{b}, —C(═O)R^{c}, —OR^{b}, NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₙR^{c} and —S(═O)ₙNR^{a}R^{a};

m is 1 or 2;

n is 0, 1 or 2; and

p is 0, 1, 2, 3, or 4;

provided that if R² is methyl, p is 0, and R⁶ is unsubstituted phenyl, then R¹ is not 2,4-dihalophenyl, 2,4-dimethylphenyl, 2,4-diethylphenyl, 2,4,6-trihalophenyl, or 2,3,4-trihalophenyl;

or a pharmaceutically acceptable salt thereof.

In one aspect, the calcimimetic compound used in the methods of the invention can be N-((6-(methyloxy)-4′-(trifluoromethyl)-1,1′-biphenyl-3-yl)methyl)-1-phenylethanamine, or a pharmaceutically acceptable salt thereof.

In one aspect, the calcimimetic compound can be cinacalcet HCl.

In one aspect, the invention provides methods of inhibiting, decreasing, or preventing vascular calcification, wherein a vitamin D sterol had been previously administered to the subject. In one aspect, the vitamin D sterol can be calcitriol, alfacalcidol, doxercalciferol, maxacalcitol or paricalcitol. In one aspect, the calcimimetic compound can be administered prior to or following administration of a vitamin D sterol. In another aspect, the calcimimetic compound can be administered in combination with a vitamin D sterol.

In one aspect, the calcimimetic compound can be administered in combination with RENAGEL®.

The invention further provides methods of decreasing serum creatinine levels in a subject, comprising administering a therapeutically effective of a calcimimetic compound to the subject. In one aspect, the subject can be suffering from increased serum creatinine levels induced by the administration of a vitamin D sterol to the subject.

### BRIEF DESCRIPTION OF THE DRAWINGS

FIG. 1 schematically represents the experimental schedule of animal treatments.

FIG. 2 schematically represents serum levels of ionized calcium in an animal model of CKD.

FIG. 3 illustrates serum levels of phosphorus in an animal model of CKD.

FIG. 4 is a schematic representation of serum levels of parathyroid hormone in an animal model of CKD.

FIG. 5 illustrates the calcium content of the aorta in an animal model of CKD.

FIG. 6 schematically represents the phosphorus content of the aorta in an animal model of CKD.

FIG. 7 represents Von Kossa stained sections of the aorta in an animal model of CKD.

FIG. 8 represents the calcium and phosphorus content of the aorta in an animal model of CKD.

FIG. 9 is a schematic representation of the experimental schedule of animal treatments in adenine-induced vascular calcification model.

FIG. 10 represents a scheme of adenine induced vascular calcification.

FIG. 11 is a schematic representation of attenuation of parathyroid hyperplasia in an animal model of CKD.

FIG. 12 is a schematic representation of change in parathyroid weights the adenine-induced CKD model with vascular calcification.

FIG. 13 demonstrates changes in serum PTH by treatment in the adenine-induced CKD model with vascular calcification.

FIG. 14 illustrates the change in aortic bone mineral density by treatment in the adenine-induced CKD model with vascular calcification.

FIG. 15 illustrates the effect of treatment on blood urea nitrogen (BUN) and creatinine in the adenine-induced CKD model with vascular calcification.

FIG. 16 demonstrates the effect of treatment on ionized calcium in the adenine-induced CKD model with vascular calcification

FIG. 17 demonstrates the effect of treatment on serum phosphorus in the adenine-induced CKD with vascular calcification.

FIG. 18 demonstrates the effect of treatment on serum Ca in the adenine-induced CKD with vascular calcification.

FIG. 19 illustrates the effect of treatment with Compound B on tissues with calcitriol-induced calcification.

FIG. 20 represents the effect of treatment with Compound B on tissues with paricalcitol-induced calcification.

### DETAILED DESCRIPTION OF THE INVENTION

I. Summary

The invention is directed to methods of reducing, inhibition, or prevention of vascular calcification.

II. Definitions

“Vascular calcification,” as used herein, means formation, growth or deposition of extracellular matrix hydroxyapatite (calcium phosphate) crystal deposits in blood vessels. Vascular calcification encompasses coronary, valvular, aortic, and other blood vessel calcification. The term includes atherosclerotic and medial wall calcification.

“Atherosclerotic calcification” means vascular calcification occurring in atheromatous plaques along the intimal layer of arteries.

“Medial calcification,” “medial wall calcification,” or “Monckeberg's sclerosis,” as used herein, means calcification characterized by the presence of calcium in the medial wall of arteries.

The term “treatment” or “treating” includes the administration, to a person in need, of an amount of a calcimimetic compound, which will inhibit, decrease or reverse development of a pathological vascular calcification condition. “Inhibiting,” in connection with inhibiting vascular calcification, is intended to mean preventing, retarding, or reversing formation, growth or deposition of extracellular matrix hydroxyapatite crystal deposits. Treatment of diseases and disorders herein is intended to also include therapeutic administration of a compound of the invention (or a pharmaceutical salt, derivative or prodrug thereof) or a pharmaceutical composition containing said compound to a subject (i.e., an animal, for example a mammal, such as a human) believed to be in need of preventative treatment, such as, for example, pain, inflammation and the like. Treatment also encompasses administration of the compound or pharmaceutical composition to subjects not having been diagnosed as having a need thereof, i.e., prophylactic administration to the subject. Generally, the subject is initially diagnosed by a licensed physician and/or authorized medical practitioner, and a regimen for prophylactic and/or therapeutic treatment via administration of the compound(s) or compositions of the invention is suggested, recommended or prescribed.

The phrase “therapeutically effective amount” is the amount of the calcimimetic compound that will achieve the goal of improvement in disorder severity and the frequency of incidence. The improvement in disorder severity includes the reversal of vascular calcification, as well as slowing down the progression of vascular calcification. In one aspect, “therapeutically effective amount” means the amount of the calcimimetic compound that decreases serum creatinine levels or prevents an increase in serum creatinine levels.

As used herein, the term “subject” is intended to mean a human or other mammal, exhibiting, or at risk of developing, calcification. Such an individual can have, or be at risk of developing, for example, vascular calcification associated with conditions such as atherosclerosis, stenosis, restenosis, renal failure, diabetes, prosthesis implantation, tissue injury or age-related vascular disease. The prognostic and clinical indications of these conditions are known in the art. An individual treated by a method of the invention can have a systemic mineral imbalance associated with, for example, diabetes, chronic kidney disease, renal failure, kidney transplantation or kidney dialysis.

Animal models that are reliable indicators of human atherosclerosis, renal failure, hyperphosphatemia, diabetes, age-related vascular calcification and other conditions associated with vascular calcification are known in the art. For example, an experimental model of calcification of the vessel wall is described by Yamaguchi et al., Exp. Path. 25: 185-190, 1984.

III. Calcimimetics Compounds and Pharmaceutical Compositions Comprising Them Administration and Dosage

As used herein, the term “calcimimetic compound” refers to a compound that binds to calcium sensing receptors and induces a conformational change that reduces the threshold for calcium sensing receptors activation by the endogenous ligand Ca2+, thereby reducing parathyroid hormone (PTH) secretion. These calcimimetic compounds can also be considered allosteric modulators of the calcium receptors.

Calcimimetic compounds useful in the present invention include those disclosed in, for example, European Patent No. 933 354 and 1 235 797; International Publication Nos. WO 01/34562, WO 93/04373, WO 94/18959, WO 95/11221, WO 96/12697, WO 97/41090; U.S. Pat. Nos. 5,688,938, 5,763,569, 5,962,314, 5,981,599, 6,001,884, 6,011,068, 6,031,003, 6,172,091, 6,211,244, 6,313,146, 6,342,532, 6,362,231, 6,432,656, 6,710,088, 6,908,935 and U.S. Patent Application Publication No. 2002/0107406.

In certain embodiments, the calcimimetic compound is chosen from compounds of Formula I and pharmaceutically acceptable salts thereof:

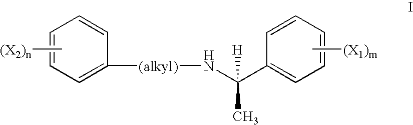

wherein:

X₁ and X₂, which may be identical or different, are each a radical chosen from CH₃, CH₃O, CH₃CH₂O, Br, Cl, F, CF₃, CHF₂, CH₂F, CF₃O, CH₃S, OH, CH₂OH, CONH₂, CN, NO₂, CH₃CH₂, propyl, isopropyl, butyl, isobutyl, t-butyl, acetoxy, and acetyl radicals, or two of X₁ may together form an entity chosen from fused cycloaliphatic rings, fused aromatic rings, and a methylene dioxy radical, or two of X₂ may together form an entity chosen from fused cycloaliphatic rings, fused aromatic rings, and a methylene dioxy radical; provided that X₂ is not a 3-t-butyl radical;

n ranges from 0 to 5;

m ranges from 1 to 5; and

the alkyl radical is chosen from C1-C3 alkyl radicals, which are optionally substituted with at least one group chosen from saturated and unsaturated, linear, branched, and cyclic C1-C9 alkyl groups, dihydroindolyl and thiodihydroindolyl groups, and 2-, 3-, and 4-piperid(in)yl groups.

The calcimimetic compound may also be chosen from compounds of Formula II: 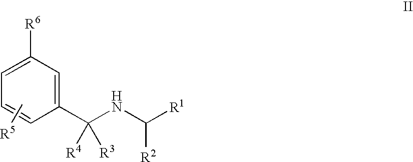
and pharmaceutically acceptable salts thereof,
wherein:

R¹ is aryl, substituted aryl, heterocyclyl, substituted heterocyclyl, cycloalkyl, or substituted cycloalkyl;

R² is alkyl or haloalkyl;

R³ is H, alkyl, or haloalkyl;

R⁴ is H, alkyl, or haloalkyl;

each R⁵ present is independently selected from the group consisting of alkyl, substituted alkyl, alkoxy, substituted alkoxy, halogen, —C(═O)OH, —CN, —NR^{d}S(═O)ₘR^{d}, —NR^{d}C(═O)NR^{d}R^{d}, —NR^{d}S(═O)ₘNR^{d}R^{d}, or —NR^{d}C(═O)R^{d};

R⁶ is aryl, substituted aryl, heterocyclyl, substituted heterocyclyl, cycloalkyl, or substituted cycloalkyl;

each R^{a} is, independently, H, alkyl or haloalkyl;

each R^{b} is, independently, aryl, aralkyl, heterocyclyl, or heterocyclylalkyl, each of which may be unsubstituted or substituted by up to 3 substituents selected from the group consisting of alkyl, halogen, haloalkyl, alkoxy, cyano, and nitro;

each R^{c} is, independently, alkyl, haloalkyl, phenyl or benzyl, each of which may be substituted or unsubstituted;

each R^{d} is, independently, H, alkyl, aryl, aralkyl, heterocyclyl, or heterocyclylalkyl wherein the alkyl, aryl, aralkyl, heterocyclyl, and heterocyclylalkyl are substituted by 0, 1, 2, 3 or 4 substituents selected from alkyl, halogen, haloalkyl, alkoxy, cyano, nitro, R^{b}, C(═O)R^{c}, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₙR^{c}and —S(═O)ₙNR^{a}R^{a};

m is 1 or 2;

n is 0, 1 or 2; and

p is 0, 1, 2, 3, or 4;

provided that if R² is methyl, p is 0, and R⁶ is unsubstituted phenyl, then R¹ is not 2,4-dihalophenyl, 2,4-dimethylphenyl, 2,4-diethylphenyl, 2,4,6-trihalophenyl, or 2,3,4-trihalophenyl. These compounds are described in detail in published US patent application number 20040082625, which is incorporated herein by reference.

In one aspect of the invention the compound of Formula II can have the formula

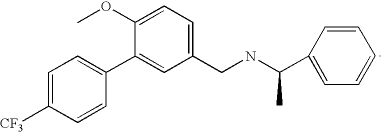

In certain embodiments of the invention the calcimimetic compound can be chosen from compounds of Formula III 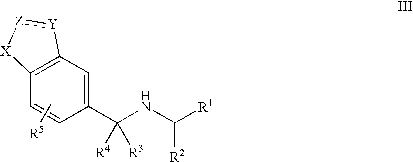
and pharmaceutically acceptable salts thereof, wherein:

[symbol] represents a double or single bond;

R¹ is R^{b};

R² is C₁₋₈ alkyl or C₁₋₄ haloalkyl;

R³ is H, C₁₋₄ haloalkyl or C₁₋₈ alkyl;

R⁴ is H, C₁₋₄ haloalkyl or C₁₋₄ alkyl;

R⁵ is, independently, in each instance, H, C₁₋₈alkyl, C₁₋₄haloalkyl, halogen, —OC₁₋₆alkyl, —NR^{a}R^{d} or NR^{d}C(═O)R^{d};

X is —CR^{d}═N—, —N═CR^{d}—, O, S or —NR^{d}—;

when [symbol] is a double bond then Y is ═CR⁶— or ═N— and Z is —CR⁷═ or —N═; and when [symbol] is a single bond then Y is —CR^{a}R⁶— or —NR^{d}— and Z is —CR^{a}R⁷— or —NR^{d}—; and

R⁶ is R^{d}, C₁₋₄haloalkyl, —C(═O)R^{c}, —OC₁₋₆alkyl, —OR^{b}, —NR^{a}R^{a}, —NR^{a}R^{b}, —C(═)OR^{c}, —C(═O)NR^{a}R^{a}, —OC(═O)R^{c}, —NR^{a}C(═O)R^{c}, cyano, nitro, —NR^{a}S(═O)ₘR^{c}or —S(═O)ₘNR^{a}R^{a};

R⁷ is R^{d}, C₁₋₄haloalkyl, —C(═O)R^{c}, —OC₁₋₆alkyl, —OR^{b}, —NR^{a}R^{a}, —NR^{a}R^{b}, —C(═O)OR^{c}, —C(═O)NR^{a}R^{a}, —OC(═O)R^{c}, —NR^{a}C(═O)R^{c}, cyano, nitro, —NR^{a}S(═O)ₘR^{c}or —S(═O)ₘNR^{a}R^{a}; or R⁶ and R⁷ together form a 3- to 6-atom saturated or unsaturated bridge containing 0, 1, 2 or 3 N atoms and 0, 1 or 2 atoms selected from S and O, wherein the bridge is substituted by 0, 1 or 2 substituents selected from R⁵; wherein when R⁶ and R⁷ form a benzo bridge, then the benzo bridge may be additionally substituted by a 3- or 4-atoms bridge containing 1 or 2 atoms selected from N and O, wherein the bridge is substituted by 0 or 1 substituents selected from C₁₋₄alkyl;

R^{a} is, independently, at each instance, H, C₁₋₄haloalkyl or C₁₋₆alkyl;

R^{b} is, independently, at each instance, phenyl, benzyl, naphthyl or a saturated or unsaturated 5- or 6-membered ring heterocycle containing 1, 2 or 3 atoms selected from N, O and S, with no more than 2 of the atoms selected from O and S, wherein the phenyl, benzyl or heterocycle are substituted by 0, 1, 2 or 3 substituents selected from C₁₋₆alkyl, halogen, C₁₋₄haloalkyl, —OC₁₋₆alkyl, cyano and nitro;

R^{c} is, independently, at each instance, C₁₋₆alkyl, C₁₋₄haloalkyl, phenyl or benzyl;

R^{d} is, independently, at each instance, H, C₁₋₆alkyl, phenyl, benzyl or a saturated or unsaturated 5- or 6-membered ring heterocycle containing 1, 2 or 3 atoms selected from N, O and S, with no more than 2 of the atoms selected from O and S, wherein the C₁₋₆ alkyl, phenyl, benzyl, naphthyl and heterocycle are substituted by 0, 1, 2, 3 or 4 substituents selected from C₁₋₆alkyl, halogen, C₁₋₄haloalkyl, —OC₁₋₆alkyl, cyano and nitro, R^{b}, —C(═O)R^{c}, —OR^{b}, NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₘR^{c}and —S(═O)ₘNR^{a}R^{a}; and

m is 1 or 2.

Compounds of Formula III are described in detail in U.S. patent application 20040077619, which is incorporated herein by reference.

In one aspect, a calcimimetic compound is N-(3-[2-chlorophenyl]-propyl)-R-α-methyl-3-methoxybenzylamine HCl (Compound A). In another aspect, a calcimimetic compound is N-((6-(methyloxy)-4′-(trifluoromethyl)-1,1′-biphenyl-3-yl)methyl)-1-phenylethanamine (Compound B).

Calcimimetic compounds useful in the method of the invention include the calcimimetic compounds described above, as well as their stereoisomers, enantiomers, polymorphs, hydrates, and pharmaceutically acceptable salts of any of the foregoing.

Calcimimetic compounds useful in the present invention can be used in the form of pharmaceutically acceptable salts derived from inorganic or organic acids. The salts include, but are not limited to, the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, mandelate, methansulfonate, nicotinate, 2-naphthalenesulfonate, oxalate, palmoate, pectinate, persulfate, 2-phenylpropionate, picrate, pivalate, propionate, salicylate, succinate, sulfate, tartrate, thiocyanate, tosylate, mesylate, and undecanoate. When compounds of the invention include an acidic function such as a carboxy group, then suitable pharmaceutically acceptable salts for the carboxy group are well known to those skilled in the art and include, for example, alkaline, alkaline earth, ammonium, quaternary ammonium cations and the like. For additional examples of “pharmacologically acceptable salts,” see infra and Berge et al. J. Pharm. Sci. 66: 1, 1977. In certain embodiments of the invention salts of hydrochloride and salts of methanesulfonic acid can be used.

In some aspects of the present invention, the calcium-receptor active compound can be chosen from cinacalcet, i.e., N-(1-(R)-(1-naphthyl)ethyl]-3-[3-(trifluoromethyl)phenyl]-1-aminopropane, cinacalcet HCl, and cinacalcet methanesulfonate. The calcimimetic compound, such as cinacalcet HCl and cinacalcet methanesulfonate, can be in various forms such as amorphous powders, crystalline powders, and mixtures thereof. The crystalline powders can be in forms including polymorphs, psuedopolymorphs, crystal habits, micromeretics, and particle morphology.

For administration, the compounds of this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

The therapeutically effective amount of the calcium receptor-active compound in the compositions disclosed herein ranges from about 1 mg to about 360 mg, for example from about 5 mg to about 240 mg, or from about 20 mg to about 100 mg of the calcimimetic compound per subject. In some aspects, the therapeutically effective amount of cinacalcet HCl or other calcimimetic compound in the composition can be chosen from about 5 mg, about 15 mg, about 20 mg, about 30 mg, about 50 mg, about 60 mg, about 75 mg, about 90 mg, about 120 mg, about 150 mg, about 180 mg, about 210 mg, about 240 mg, about 300 mg, or about 360 mg.

While it may be possible to administer a calcimimetic compound to a subject alone, the compound administered will normally be present as an active ingredient in a pharmaceutical composition. Thus, a pharmaceutical composition of the invention may comprise a therapeutically effective amount of at least one calcimimetic compound, or an effective dosage amount of at least one calcimimetic compound.

As used herein, an “effective dosage amount” is an amount that provides a therapeutically effective amount of the calcimimetic compound when provided as a single dose, in multiple doses, or as a partial dose. Thus, an effective dosage amount of the calcimimetic compound of the invention includes an amount less than, equal to or greater than an effective amount of the compound; for example, a pharmaceutical composition in which two or more unit dosages, such as in tablets, capsules and the like, are required to administer an effective amount of the compound, or alternatively, a multidose pharmaceutical composition, such as powders, liquids and the like, in which an effective amount of the calcimimetic compound is administered by administering a portion of the composition.

Alternatively, a pharmaceutical composition in which two or more unit dosages, such as in tablets, capsules and the like, are required to administer an effective amount of the calcimimetic compound may be administered in less than an effective amount for one or more periods of time (e.g., a once-a-day administration, and a twice-a-day administration), for example to ascertain the effective dose for an individual subject, to desensitize an individual subject to potential side effects, to permit effective dosing readjustment or depletion of one or more other therapeutics administered to an individual subject, and/or the like.

The effective dosage amount of the pharmaceutical composition disclosed herein ranges from about 1 mg to about 360 mg from a unit dosage form, for example about 5 mg, about 15 mg, about 30 mg, about 50 mg, about 60 mg, about 75 mg, about 90 mg, about 120 mg, about 150 mg, about 180 mg, about 210 mg, about 240 mg, about 300 mg, or about 360 mg from a unit dosage form.

In some aspects of the present invention, the compositions disclosed herein comprise a therapeutically effective amount of a calcimimetic compound for the treatment or prevention of vascular calcification. For example, in certain embodiments, the calcimimetic compound such as cinacalcet HCl can be present in an amount ranging from about 1% to about 70%, such as from about 5% to about 40%, from about 10% to about 30%, or from about 15% to about 20%, by weight relative to the total weight of the composition.

The compositions of the invention may contain one or more active ingredients in addition to the calcimimetic compound. The additional active ingredient may be another calcimimetic compound, or it may be an active ingredient having a different therapeutic activity. Examples of such additional active ingredients include, for example, vitamins and their analogs, such as vitamin D and analogs thereof (including vitamin D sterols such as calcitriol, alfacalcidol, doxercalciferol, maxacalcitol and paricalcitol), antibiotics, lanthanum carbonate, lipid-lowering agents, such as LIPITOR®, anti-hypertensives, anti-inflammatory agents (steroidal and non-steroidal), inhibitors of pro-inflammatory cytokine (ENBREL®, KINERET®), and cardiovascular agents. When administered as a combination, the therapeutic agents can be formulated as separate compositions that are given at the same time or different times, or the therapeutic agents can be given as a single composition.

In one aspect of combination therapy, the compositions of the invention may be used with vitamin D sterols and/or RENAGEL®. In one aspect, the compositions of the invention may be administered before administration of vitamin D sterols and/or RENAGEL®. In another aspect, the compositions of the invention can be administered concurrently with vitamin D sterols and/or RENAGEL®. In a further aspect, the compositions of the invention can be administered after administration of vitamin D sterols and/or RENAGEL®. The dosage regimen for treating a disease condition with the combination therapy of this invention is selected in accordance with a variety of factors, including the type, age, weight, sex and medical condition of the patient, the severity of the disease, the route of administration, and the particular compound employed, and thus may vary widely.

IV. Assessment of Vascular Calcification

Methods of detecting and measuring vascular calcification are well known in the art. In one aspect, methods of measuring calcification include direct methods of detecting and measuring extent of calcium-phosphorus depositions in blood vessels.

In one aspect, direct methods of measuring vascular calcification comprise in vivo imaging methods such as plain film roentgenography, coronary arteriography; fluoroscopy, including digital subtraction fluoroscopy; cinefluorography; conventional, helical, and electron beam computed tomography; intravascular ultrasound (IVUS); magnetic resonance imaging; and transthoracic and transesophageal echocardiography. Fluoroscopy and EBCT are most commonly used to detect calcification noninvasively, while cinefluorography and IVUS are used by coronary interventionalists to evaluate calcification in specific lesions before angioplasty.

In one aspect, vascular calcification can be detected by plain film roentgenography. The advantage of this method is availability of the film and the low cost of the method, however, the disadvantage is its low sensitivity. Kelley M. & Newell J. Cardiol Clin. 1: 575-595, 1983.

In another aspect, fluoroscopy can be used to detect calcification in coronary arteries. Although fluoroscopy can detect moderate to large calcifications, its ability to identify small calcific deposits is low. Loecker et al. J Am Coll Cardiol. 19: 1167-1172, 1992. Fluoroscopy is widely available in both inpatient and outpatient settings and is relatively inexpensive, but it has several disadvantages. In addition to only a low to moderate sensitivity, fluoroscopic detection of calcium is dependent on the skill and experience of the operator as well as the number of views studied. Other important factors include variability of fluoroscopic equipment, the patient's body habitus, overlying anatomic structures, and overlying calcifications in structures such as vertebrae and valve annuli. With fluoroscopy, quantification of calcium is not possible, and film documentation is not commonly obtained.

In yet another aspect, vascular detection can be detected by conventional computed tomography (CT). Because calcium attenuates the x-ray beam, computed tomography (CT) is extremely sensitive in detecting vascular calcification. While conventional CT appears to have better capability than fluoroscopy to detect coronary artery calcification, its limitations are slow scan times resulting in motion artifacts, volume averaging, breathing misregistration, and inability to quantify amount of plaque. Wexler et al. Circulation 94: 1175-1192, 1996.

In a further aspect, calcification can be detected by helical or spiral computer tomography, which has considerably faster scan times than conventional CT. Overlapping sections also improve calcium detection. Shemesh et al. reported coronary calcium imaging by helical CT as having a sensitivity of 91% and a specificity of 52% when compared with angiographically significant coronary obstructive disease. Shemesh et al. Radiology 197: 779-783, 1995. However, other preliminary data have shown that even at these accelerated scan times, and especially with single helical CT, calcific deposits are blurred due to cardiac motion, and small calcifications may not be seen. Baskin et al. Circulation 92(suppl I): 1-651, 1995. Thus, helical CT remains superior to fluoroscopy and conventional CT in detecting calcification. Double-helix CT scanners appear to be more sensitive than single-helix scanners in detection of coronary calcification because of their higher resolution and thinner slice capabilities. Wexler et al., supra.

In another aspect, Electron Beam Computed Tomography (EBCT) can be used for detection of vascular calcification. EBCT uses an electron gun and a stationary tungsten “target” rather than a standard x-ray tube to generate x-rays, permitting very rapid scanning times. Originally referred to as cine or ultrafast CT, the term EBCT is now used to distinguish it from standard CT scans because modern spiral scanners are also achieving subsecond scanning times. For purposes of detecting coronary calcium, EBCT images are obtained in 100 ms with a scan slice thickness of 3 mm. Thirty to 40 adjacent axial scans are obtained by table incrementation. The scans, which are usually acquired during one or two separate breath-holding sequences, are triggered by the electrocardiographic signal at 80% of the RR interval, near the end of diastole and before atrial contraction, to minimize the effect of cardiac motion. The rapid image acquisition time virtually eliminates motion artifact related to cardiac contraction. The unopacified coronary arteries are easily identified by EBCT because the lower CT density of periarterial fat produces marked contrast to blood in the coronary arteries, while the mural calcium is evident because of its high CT density relative to blood. Additionally, the scanner software allows quantification of calcium area and density. An arbitrary scoring system has been devised based on the x-ray attenuation coefficient, or CT number measured in Hounsfield units, and the area of calcified deposits. Agatston et al. J Am Coll Cardiol. 15:827-832, 1990. A screening study for coronary calcium can be completed within 10 or 15 minutes, requiring only a few seconds of scanning time. Electron beam CT scanners are more expensive than conventional or spiral CT scanners and are available in relatively fewer sites.

In one aspect, intravascular ultrasound (IVUS) can be used for detecting vascular calcification, in particular, coronary atherosclerosis. Waller et al. Circulation 85: 2305-2310, 1992. By using transducers with rotating reflectors mounted on the tips of catheters, it is possible to obtain cross-sectional images of the coronary arteries during cardiac catheterization. The sonograms provide information not only about the lumen of the artery but also about the thickness and tissue characteristics of the arterial wall. Calcification is seen as a hyperechoic area with shadowing: fibrotic noncalcified plaques are seen as hyperechoic areas without shadowing. Honye et al. Trends Cardiovasc Med. 1: 305-311, 1991. The disadvantages in use of IVUS, as opposed to other imaging modalities, are that it is invasive and currently performed only in conjunction with selective coronary angiography, and it visualizes only a limited portion of the coronary tree. Although invasive, the technique is clinically important because it can show atherosclerotic involvement in patients with normal findings on coronary arteriograms and helps define the morphological characteristics of stenotic lesions before balloon angioplasty and selection of atherectomy devices. Tuzcu et al. J Am Coll Cardiol. 27: 832-838, 1996.

In another aspect, vascular calcification can be measured by magnetic resonance imaging (MRI). However, the ability of MRI to detect coronary calcification is somewhat limited. Because microcalcifications do not substantially alter the signal intensity of voxels that contain a large amount of soft tissue, the net contrast in such calcium collections is low. Therefore, MRI detection of small quantities of calcification is difficult, and there are no reports or expected roles for MRI in detection of coronary artery calcification. Wexler et al., supra.

In another aspect, vascular calcification can be measured by transthoracic (surface) echocardiography, which is particularly sensitive to detection of mitral and aortic valvular calcification; however, visualization of the coronary arteries has been documented only on rare occasions because of the limited available external acoustic windows. Transesophageal echocardiography is a widely available methodology that often can visualize the proximal coronary arteries. Koh et al. Int J Cardiol. 43: 202-206, 1994. Fernandes et al. Circulation 88: 2532-2540, 1993.

In another aspect, vascular calcification can be assessed ex vivo by Van Kossa method. This method relies upon the principle that silver ions can be displaced from solution by carbonate or phosphate ions due to their respective positions in the electrochemical series. The argentaffin reaction is photochemical in nature and the activation energy is supplied from strong visible or ultra-violet light. Since the demonstrable forms of tissue carbonate or phosphate ions are invariably associated with calcium ions the method may be considered as demonstrating sites of tissue calcium deposition.

Other methods of direct measuring calcification may include, but not limited to, immunofluorescent staining and densitometry. In another aspect, methods of assessing vascular calcification include methods of measuring determinants and/or risk factors of vascular calcification. Such factors include, but are not limited to, serum levels of phosphorus, calcium, and calcium×phosphorus product, parathyroid hormone (PTH), low-density lipoprotein cholesterol (LDL), high-density lipoprotein cholesterol (HDL), tryglycerides, and creatinine. Methods of measuring these factors are well known in the art. Other methods of assessing vascular calcification include assessing factors of bone formation. Such factors include bone formation markers such as bone-specific alkaline phosphatase (BSAP), osteocalcin (OC), carboxyterminal propeptide of type I collagen (PICP), and aminoterminal propeptide of type I collagen (PINP); serum bone resorption markers such as cross-linked C-telopeptide of type I collagen (ICTP), tartrate-resistant acid phosphatase, TRACP and TRAP5B, N-telopeptide of collagen cross-links (NTx), and C-telopeptide of collagen cross-links (CTx); and urine bone resorption markers, such as hydroxyproline, free and total pyridinolines (Pyd), free and total deoxypyridinolines (Dpd), N-telopeptide of collagen cross-links (NTx), and C-telopeptide of collagen cross-links (CTx).

V. Methods of Treatment

In one aspect, the invention provides a method of inhibiting, decreasing or preventing vascular calcification in an individual. The method comprises administering to the individual a therapeutically effective amount of the calcimimetic compound of the invention. In one aspect, administration of the compound of the invention retards or reverses the formation, growth or deposition of extracellular matrix hydroxyapatite crystal deposits. In another aspect of the invention, administration of the compound of the invention prevents the formation, growth or deposition of extracellular matrix hydroxyapatite crystal deposits.

Methods of the invention may be used to prevent or treat atherosclerotic calcification and medial calcification and other conditions characterized by vascular calcification. In one aspect, vascular calcification may be associated with chronic renal insufficiency or end-stage renal disease. In another aspect, vascular calcification may be associated with pre- or post-dialysis or uremia. In a further aspect, vascular calcification may be associated with diabetes mellitus I or II. In yet another aspect, vascular calcification may be associated with a cardiovascular disorder.

In one aspect, administration of an effective amount of calcimimetics can reduce serum PTH without causing aortic calcification. In another aspect, administration of calcimimetics can reduce serum creatinine level or can prevent increase of serum creatinine level. In another aspect, administration of calcimimetics can attenuates parathyroid (PT) hyperplasia.

Calcimimetics may be administered alone or in combination with other drugs for treating vascular calcification, such as vitamin D sterols and/or RENAGEL®. Vitamin D sterols can include calcitriol, alfacalcidol, doxercalciferol, maxacalcitol or paricalcitol. In one aspect, calcimimetic compounds can be administered before or after administration of vitamin D sterols. In another aspect, calcimimetics can be co-administered with vitamin D sterols. The methods of the invention can be practiced to attenuate the mineralizing effect of calcitriol on vascular tissue. In one aspect, the methods of the invention can be used to reverse the effect of calcitriol of increasing the serum levels of calcium, phosphorus and Ca×P product thereby preventing or inhibiting vascular calcification. In another aspect, the methods of the invention can be used to stabilize or decrease serum creatinine levels. In one aspect, in addition to creatinine level increase due to a disease, a further increase in creatinine level can be due to treatment with vitamin D sterols such as calcitriol.

In addition, calcimimetics may be administered in conjunction with surgical and non-surgical treatments. In one aspect, the methods of the invention can be practiced in injunction with dialysis.

The following examples are offered to more fully illustrate the invention, but are not to be construed as limiting the scope thereof.

### EXAMPLE 1

This example demonstrates that the calcimimetic N-(3-[2-chlorophenyl]-propyl)-R-α-methyl-3-methoxybenzylamine HCl (Compound A) reduced serum PTH in uremic rats with secondary hyperparathyroidism (HPT) without causing aortic calcification and attenuated the mineralizing effect of calcitriol on vascular tissue.

Animals

Male Wistar rats weighing 250 g were purchased from the Animal Breeding Facility of the University of Cordoba (Spain). Rats were housed with a 12 hr/12 hr light/dark cycle and given ad libitum access to normal diet (calcium=0.9%, phosphorus=0.6%). The experimental protocols were reviewed and approved by the Ethics Committee for Animal Research of the Universidad de Cordoba (Spain), and all animals received humane care in compliance with the Principles of Laboratory Animal Care, formulated by the National Society for Medical Research and the Guide for the Care and Use of Laboratory Animals prepared by the National Academy of Science.

5/6 Nephrectomy

The rodent model of CKD used in these studies was induced by 5/6 nephrectomy (5/6 Nx), a two-step procedure that reduces the original functional renal mass by five-sixths (5/6). In the first step, animals were anesthetized using xylazine (5 mg/kg, ip) and ketamine (80 mg/kg, ip), a 5-8 mm incision was made on the left medio-lateral surface of the abdomen, and the left kidney was exposed. The left renal artery was visualized and 2 of the 3 branches tightly ligated, after which the kidney was inspected for infarct and returned to an anatomically neutral position within the peritoneal cavity. The abdominal wall and skin incisions were closed with suture, and the rat placed back into its home cage. After 1 week of recovery, the animal was reanesthetised and a 5-8 mm incision was made on the right medio-lateral surface of the abdomen. The right kidney was exposed and unencapsulated, the renal pedicle clamped and ligated, and the kidney was removed. The ligated pedicle was returned to a neutral anatomical position and the abdomen and skin incisions closed with suture materials. The animal was allowed to recover in its home cage. Sham-operated animals underwent the same procedures without renal manipulation.

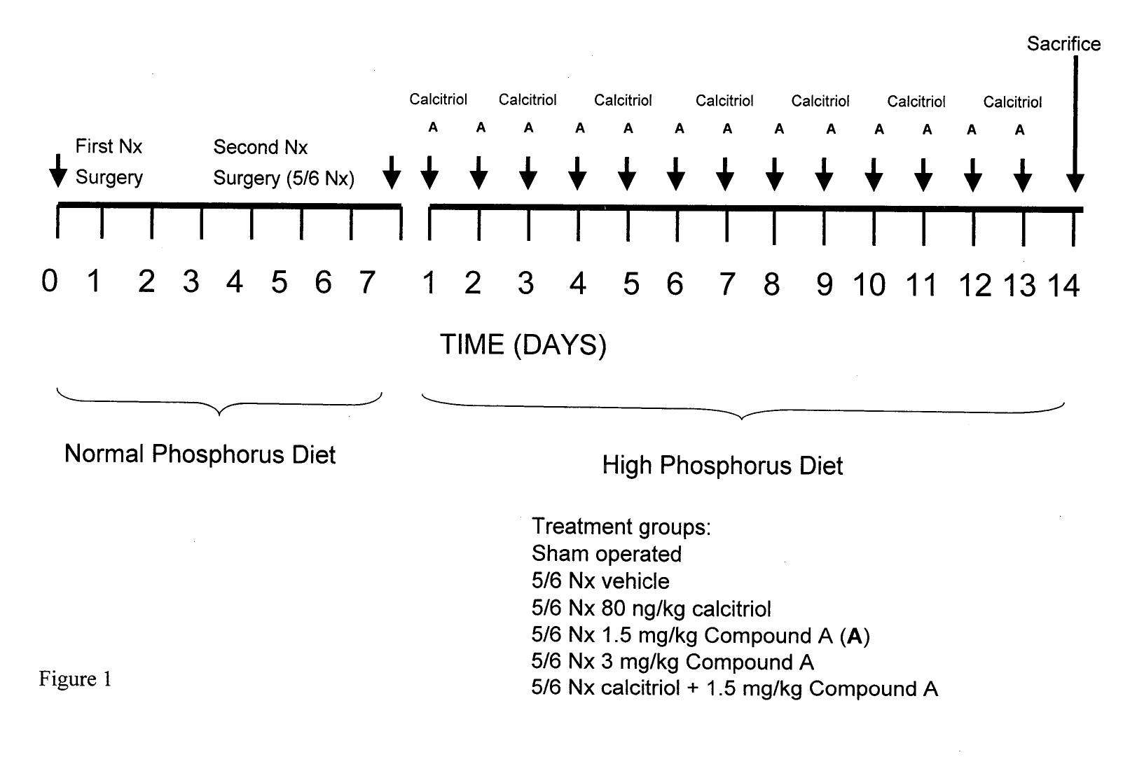

The experimental schedule is shown in FIG. 1. After the second surgery, the diet was changed to one with decreased calcium (0.6%) and increased phosphorus (0.9%) content. The rats were randomized (based on the normal distribution of baseline body weights) into 6 experimental groups: sham-operated (n=13) (used as a control), 5/6 Nx+vehicle (saline) (n=10), 5/6 Nx+calcitriol 80 ng/kg (Calcijex, Abbot) ip every other day (n=10), 5/6 Nx+Compound A 1.5 mg/kg per day sc (n=10) (Amgen, Thousand Oaks, Calif. USA), 5/6 Nx+Compound A 3 mg/kg per day sc (n=10), or 5/6 Nx+combination calcitriol 80 ng/kg and Compound A 1.5 mg/kg (n=10) dosed as above. Treatments were maintained for 14 days. The rats were sacrificed by aortic puncture and exsanguination under general anesthesia (ip sodium thiopenthal) 24 hours after the last dose of drug.

Blood Chemistries

Blood for chemistry analyses was collected from the abdominal aorta at the end of the treatment period. Blood for measurements of ionized calcium levels was collected in heparinized syringes and immediately analyzed using a Ciba-Corning 634 ISE Ca++/pH Analyzer (Ciba-Corning Essex, England). Afterwards, plasma was separated by centrifugation and stored at −70° C. until assayed. PTH levels were quantified according to the vendor's instructions using a rat PTH₍₁₋₃₄₎ immunoradiometric assay kit (Immunotopics, San Clemente, CA). Serum creatinine, phosphorous, and total calcium were measured by spectrophotometry (Sigma Diagnostics, St. Louis, Mo., USA).

Ex Vivo Assessment of Vascular Calcification

Following sacrifice, the abdominal aortas were dissected and divided in two parts. One part was fixated in 10% buffered formalin and subsequently sectioned and stained for mineralization by the von Kossa method. The other was dimineralized in 10% formic acid, and the arterial tissue calcium and phosphorous content measured in the supernatant according to the method described by Price et al Arterioscler Thromb Vasc Biol 20:317-27, 2000.

Statistics

Values were expressed as the mean±standard error (SE). The difference between means for two different groups was determined by t test; the difference between means for three or more groups was assessed by ANOVA. P<0.05 was considered significant.

Creatinine

Mean serum creatinine concentration in sham rats was 0.53±0.02 mg/dl. As expected, all 5/6 Nx rats had significantly (P<0.05) higher creatinine levels (0.83±0.04 to 0.89±0.03 mg/dl) before any drug treatment with no significant intergroup differences. Treatment with calcitriol resulted in a further significant (P<0.05) increase in serum creatinine levels (1.05±0.07 mg/dl) in relation to the other 5/6 Nx groups. The combination of calcitriol and Compound A did not significantly elevate creatinine levels (0.93±0.05 mg/dl) when compared to Compound A or vehicle-treated 5/6 Nx animals. The inclusion of Compound A with calcitriol decreased calcitriol mediated increase in serum creatinine levels.

Serum Biochemical Parameters

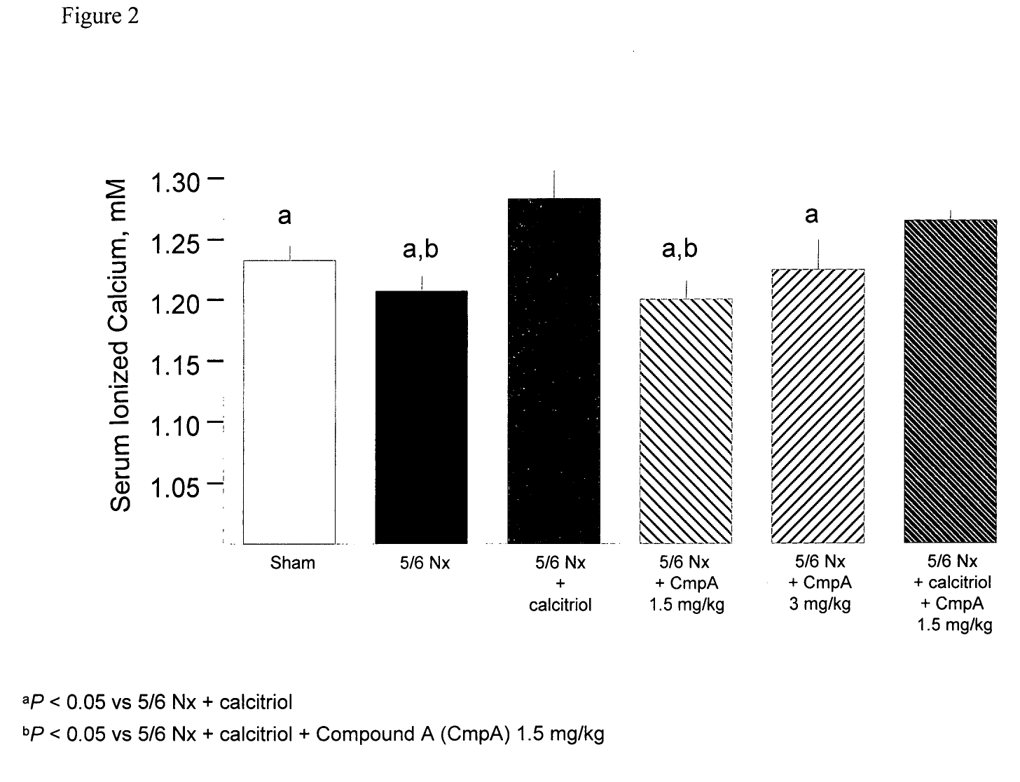

Serum levels of ionized calcium, phosphorus and PTH are depicted in FIGS. 2-4. Serum ionized calcium levels were similar in 5/6 Nx and sham groups (1.21±0.01 mmol/l vs 1.23±0.01 mmol/l). Serum ionized calcium levels in rats treated with Compound A at 1.5 (1.20±0.02 mmol/l) or 3 mg/kg (1.22±0.02 mmol/l) were not different from the 5/6 Nx vehicle-treated group (1.21±0.01 mmol/l). However, treatment with calcitriol alone or in combination with Compound A resulted in significantly (P<0.05) higher serum ionized calcium levels (1.28±0.02 mmol/l, and 1.26±0.01 mmol/l, respectively) when compared to the 5/6 Nx vehicle-treated or Compound A alone-treated groups (FIG. 2).

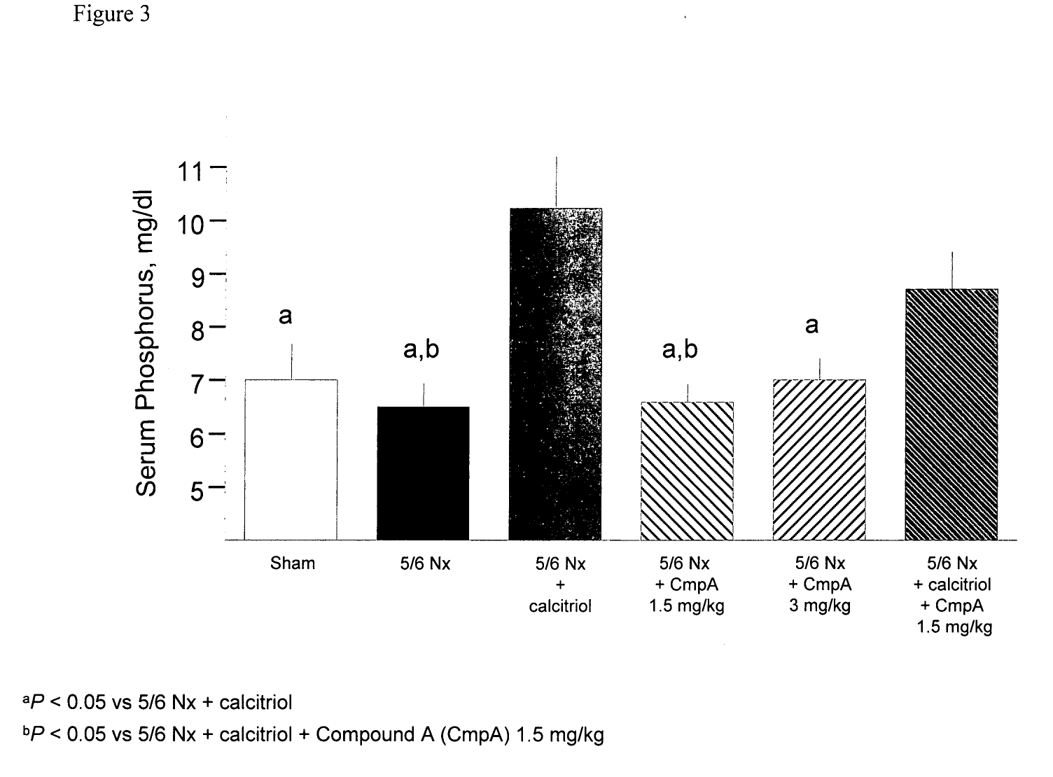

Serum phosphorus levels (FIG. 3) were not different between the sham (6.9±0.7 mg/dl), and the 5/6 Nx animals treated with vehicle (6.5±0.4 mg/dl) or Compound A at 1.5 (6.6±0.3 mg/dl) or 3 mg/kg (6.9±0.4 mg/dl). Animals that received calcitriol alone exhibited significantly (P<0.05) elevated serum phosphorous levels (10.2±0.9 mg/dl) when compared to vehicle-treated 5/6 Nx animals. The combination of Compound A and calcitriol did tend toward decreased serum phosphorus levels (8.7±0.7 mg/dl), but was still significantly (P<0.05) higher than vehicle-treated 5/6 Nx animals.

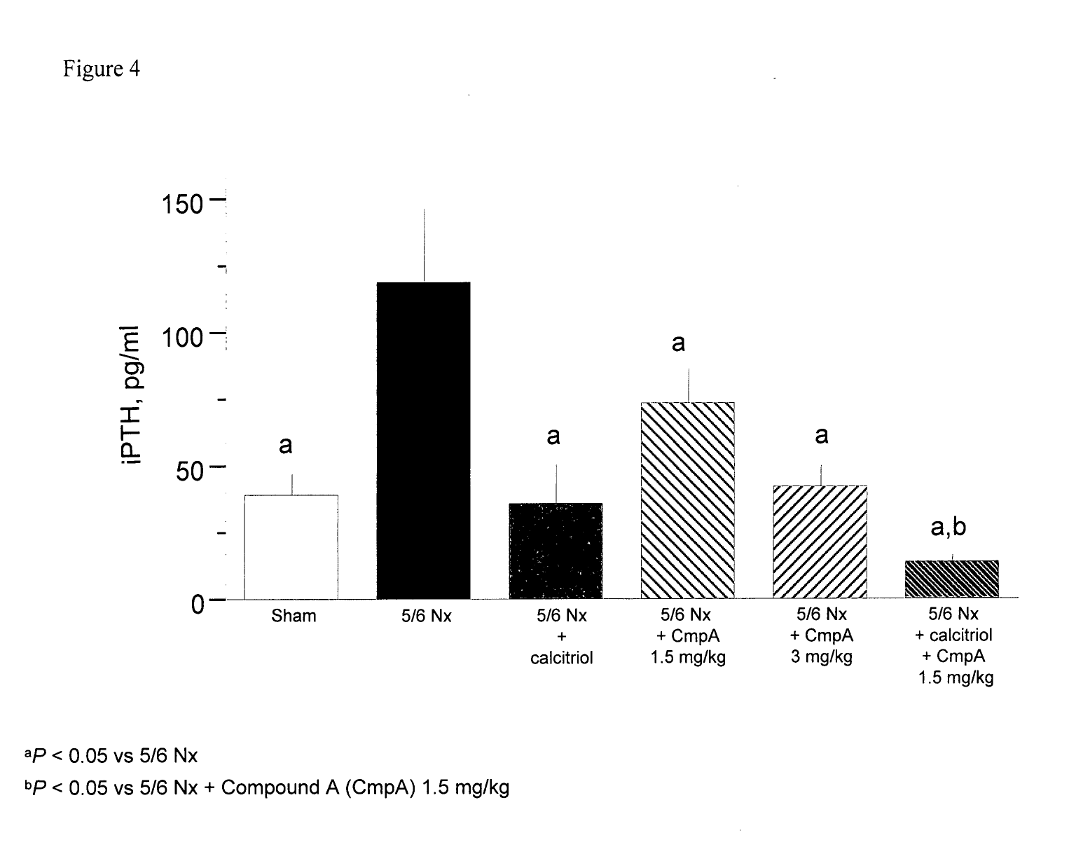

Serum PTH concentration was significantly (P<0.05) increased in 5/6 Nx rats (118.7±27.7 pg/ml), when compared to sham-operated animals (39.3±7.9 pg/ml). All the treatments employed reduced serum PTH concentrations to levels that were not significantly different from the sham rats. However, the combination of calcitriol and Compound A resulted in a significantly more (P<0.05) effective PTH suppression (13.8±2.6 pg/ml) than Compound A 1.5 mg/kg (73.5±12.8 pg/ml) alone (FIG. 4).

Aortic Mineral Content

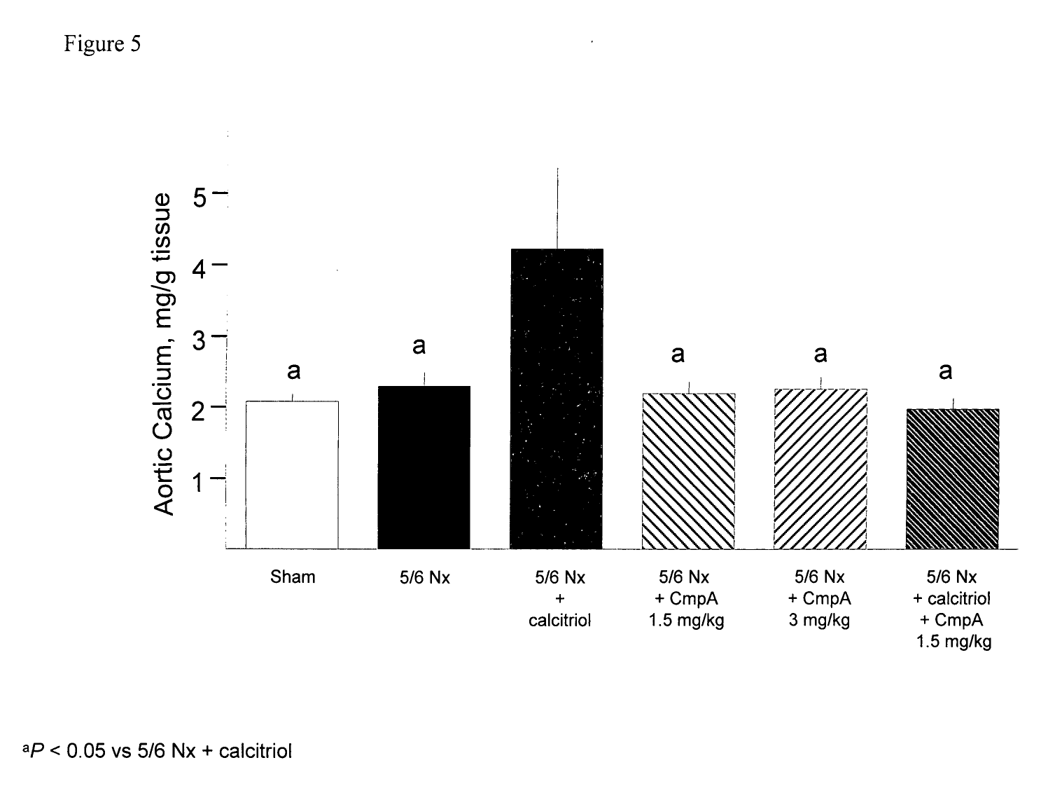

In line with the increased serum mineral levels observed with calcitriol administration, treatment of 5/6 Nx rats with calcitriol significantly (P=0.009) increased aortic calcium content (4.2±1.1 mg/g tissue) compared to vehicle-treated 5/6 Nx animals (2.3±0.2 mg/g tissue) (FIG. 5). Treatment with Compound A, however, resulted in similar aortic calcium content to vehicle-treated 5/6 Nx (P=0.882) or sham-operated animals (P=0.777) (FIG. 5). Surprisingly, given the nonsignificant effect of combination calcitriol and Compound A on serum calcium levels, the calcitriol-induced increase in aortic calcium was significantly (P=0.002) attenuated by concurrent treatment with Compound A 1.5 mg/kg (FIG. 5).

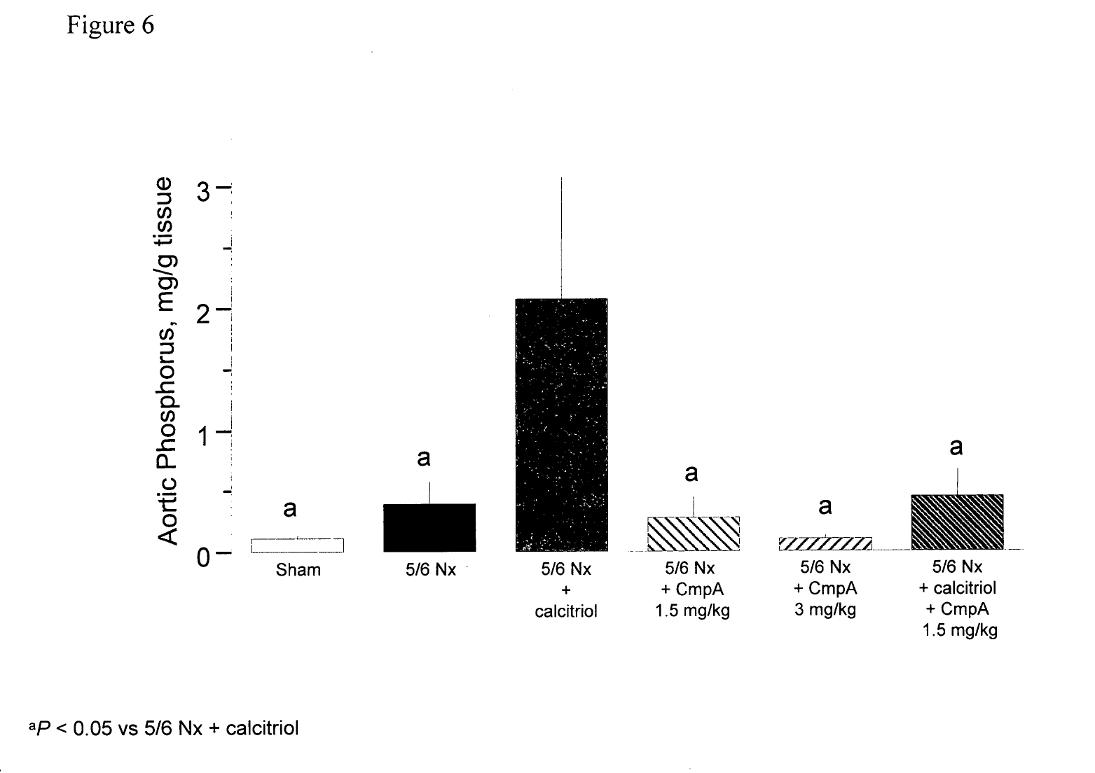

Additional analyses demonstrated that the phosphorus content of sham animals was not different from that in vehicle-treated 5/6 Nx animals (FIG. 6). Treatment of 5/6 Nx animals with calcitriol significantly (P=0.01) raised phosphorus (2.1±1 mg/g tissue) content in the aortic tissue, whereas Compound A (1.5 or 3 mg/kg) did not (FIG. 6). The calcitriol-induced increase in aortic phosphorus was significantly (P=0.013) attenuated by concurrent treatment with Compound A 1.5 mg/kg (FIG. 6).

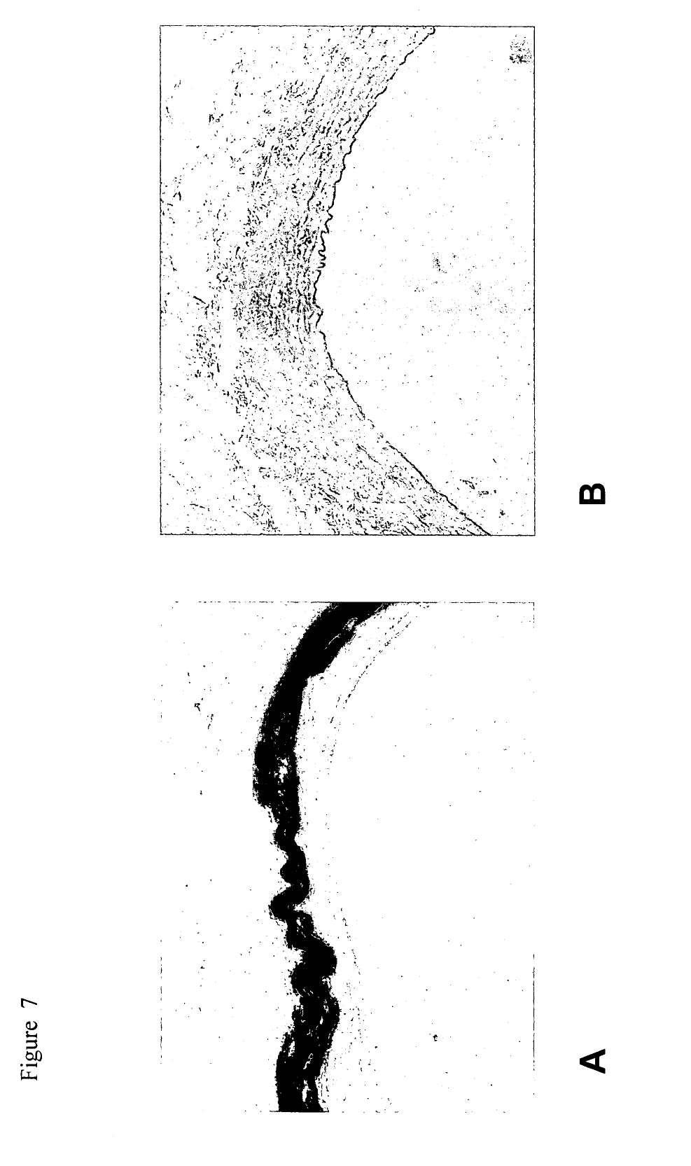

In situ aortic mineralization was examined by means of the von Kossa staining method (FIG. 7). Mineral deposits in the aorta were not observed in the either the sham, vehicle or Compound A—treated 5/6 Nx groups. However, marked von Kossa staining was detected in the media of 5/6 Nx rats treated with calcitriol alone (FIG. 7A). Interestingly, the addition of Compound A 1.5 mg/kg to the calcitriol-treatment regimen prevented the development of aortic calcification (FIG. 7B).

Vascular Calcification Regression

Animals were 5/6 Nx as previously described and placed on a high phosphorus diet (0.6% Ca; 1.2% P) for 14 days. Animals were divided into four groups (A,B,C and D n=4-5 animals per group), one group (A) received vehicle (saline 0.2 ml i.p.) while the remaining three groups (B,C and D) received calcitriol (80 ng/kg every other day i.p.) for the course of the study (28 days). On day 14 two groups (C and D) were converted to normal diet (Ca 0.9%; P 0.6%) and group C was administered Compound A at 3 mg/kg daily p.o.), while group D received vehicle (0.5 ml of 10% captisol in water p.o.) daily. The remaining groups (A and B) were kept on the high phosphorus diet and administered either calcitriol or vehicle for the course of the study (28 days). On day 28 all animals were sacrificed (CO₂) and the aortas removed for determining aortic P and Ca content (mg/g of tissue) as previously described

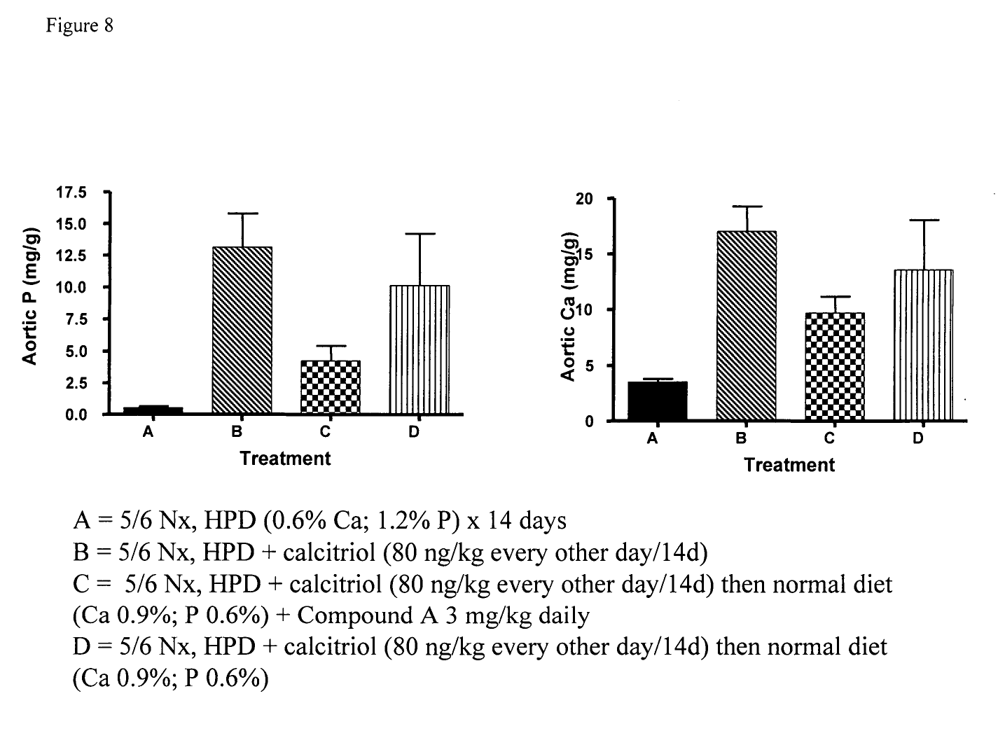

Animals that were on the high phosphorus diet and received calcitriol over the entire 28 days (Group B), showed significant (p<0.05) increases in aortic calcium and phosphorus content when compared to rats on high phosphorus diet receiving vehicle (Group A) (see FIG. 8). This indicates that calcitriol mediates increases in aortic calcium and phosphorus content (vascular calcification).

Changing the diet from a high phosphorus (group B) to a normal phosphorus diet caused a slight non-significant decrease in aortic calcium and phosphorus content (Group D). This would suggest that a diet low in phosphorus could prevent progression and/or reverse the vascular calcification process (CR1 and ESRD patients are told to eat a low phosphorus diet). Animals changed from the high phosphorus diet to a normal phosphorus diet and received Compound A 3 mg/kg while still receiving calcitriol (group C) at the end of the study demonstrated a significant (p<0.05) reduction in aortic phosphorus content and lower, aortic calcium content when compared to group D. This suggests that established vascular calcification can be reversed by a calcimimetic agent like Compound A.

### EXAMPLE 2

This example demonstrates that the calcimimetic N-((6-(methyloxy)-4′-(trifluoromethyl)-1,1′-biphenyl-3-yl)methyl)-1-phenylethanamine (Compound B) attenuates parathyroid (PT) hyperplasia, decreases serum PTH and reduces aortic vascular calcification in an animal model of CKD.

Male Sprague-Dawley rats (Charles River Laboratories) weighing 300-350 grams were used in these studies. All animals received standard lab chow (Harlan Teklad, Madison, Wis.) prior to the start of the studies. The standard lab chow was changed to a standard rodent lab chow that contained 0.75% adenine. Animals received food and water ad libitum. The animal protocol was approved by the Institutional Animal Care and Use Committee of Amgen Inc. (Thousand Oaks, Calif.).

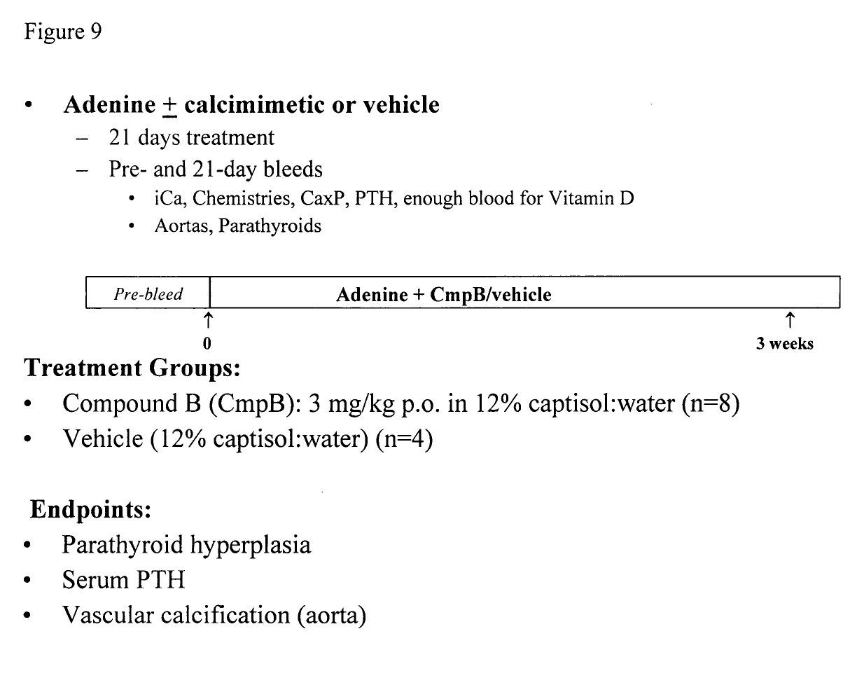

Animals were fed the adenine containing chow for 21 days, and prior to the start of the adenine diet animals were pre-bleed for baseline measurements of ionized calcium, PTH, BUN and creatinine and phosphorus levels (FIG. 9).

Blood Collection for PTH and Serum Chemistry Profile

Prior to placing animals on an adenine diet or administration of Compound B or vehicle baseline determininations of serum P, Ca, BUN, PTH and creatinine were performed. Blood was removed from the orbital sinus under while the rats were under anesthetisa (2% isoflurane in O₂). After the time 0 bleed, animals were placed on the adenine containing chow and were administered Compound B at 3 mg/kg p.o. or vehicle (12% captisol in water) daily for 21 days. At the end of the study (day 21) animals were sacrificed and the aorta and parathyroid glands were removed for histopathological analysis. Blood was removed for blood chemistries and PTH determinations. For measurement of blood ionized calcium levels, blood was collected from the abdominal aorta under anesthesthia (2% isofluorane in O₂), prior to sacrifice, with heparinized capillary tubes and analyzed using a Ciba-Corning 634 ISE Ca⁺⁺/pH Analyzer (Ciba-Corning Diagnostics Corp, Medfield, Mass.). Separately, blood was collected for PTH, blood urea nitrogen (BUN), creatinine, and serum phosphorus levels into SST (clot activator) brand blood tubes (BD, Franklin Lakes, N.J.) and allowed to clot. Serum was removed and stored at −70° C. until assayed. PTH levels were quantified according to the vendor's instructions using a rat PTH₍₁₋₃₄₎ immunoradiometric assay kit (Immutopics, San Clemente, CA). BUN, creatinine, and phosphorus levels were determined using a blood chemistry analyzer (Olympus AU 400, Melville, N.Y.).

PCNA Immunohistochemistry:

Hyperplasia was determined using parathyroid weight and proliferating cell nuclear antigen (PCNA) immunochemistry. The laryngo-tracheal complex was removed at sacrifice and stored 2-3 days in Zn-buffered formalin, then transferred to 70% alcohol and trimmed. At trimming, the parathyroids were dissected away from the thyroid and blotted dry on a lint-free Kim wipe (Kimberly Clark Corp., Roswell, Ga.) prior to being individually weighed on a Sartorius BP211D balance (Goettingen, Germany). Parathyroids were then processed for paraffin embedment. After embedding, 5 μm sections were cut and placed onto charged slides (VWR Scientific, West Chester Pa.). Immunostaining was performed on the sections according to the vendor's instructions using a PCNA staining kit (Zymed Laboratories, Inc., S. San Francisco, Calif.).

Parathyroid area was determined through the use of an area-measurement graticle containing a series of 0.01 mm² grids (area initially determined using a calibrated graticle) overlaying the central region of a parathyroid section. Sections were taken from approximately the same level of individual parathyroids. Tissue samples were visualized at 100× on a Leitz Laborlux microscope, and the number of grids overlaying the parathyroid tissue was counted. The total area of the parathyroid was thus determined by multiplying the number of grids by 0.01 mm², after which the number of PCNA positive cells in the gridded sectional area were counted and expressed as the number of PCNA positive cells/mm². Slides were coded and an observer who was unaware of treatment group assignment performed quantification of parathyroid proliferation.

Methods for Aortic Vascular Calcification

At the end of the study animals were sacrificed (CO₂), aortas removed and blood collected for P, Ca, BUN and creatinine determinations as previously described. Aortas were collected and fixed in 10% Neutral Buffered Formalin for 3-7 days, then transferred to 70% Ethanol. Bone Mineral Density (BMD; g/cm2) analysis was performed on a Lunar PIXImus 2 densitometer (Lunar PIXImus; Madison, Wis.) with the following parameter: run time 4 min. The results were analyzed using Lunar piximus software version 2.0.

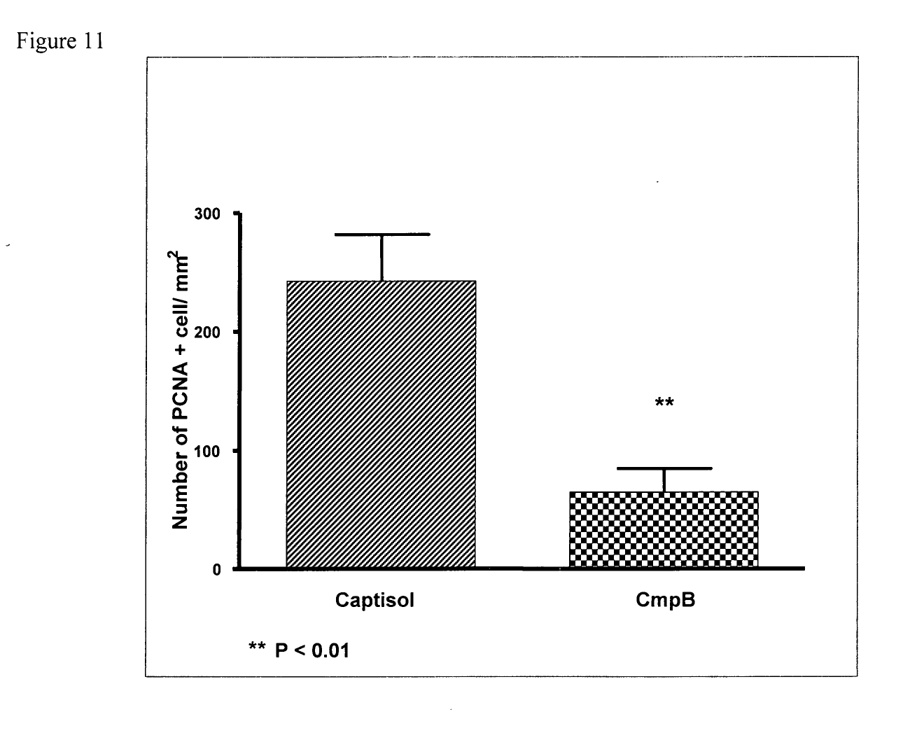

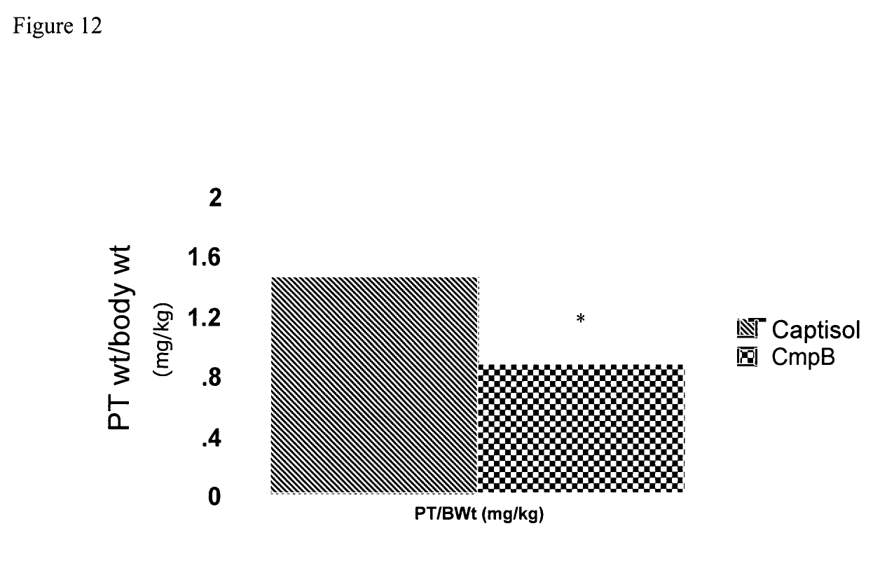

Administration of Compound B for 3 weeks significantly (p<0.01) reduced the number of PCNA-positive cells compared with vehicle-treated animals (FIG. 11). Similarly, parathyroid weights were also significantly decreased in Compound B—treated animals when compared to vehicle treated animals (FIG. 12).

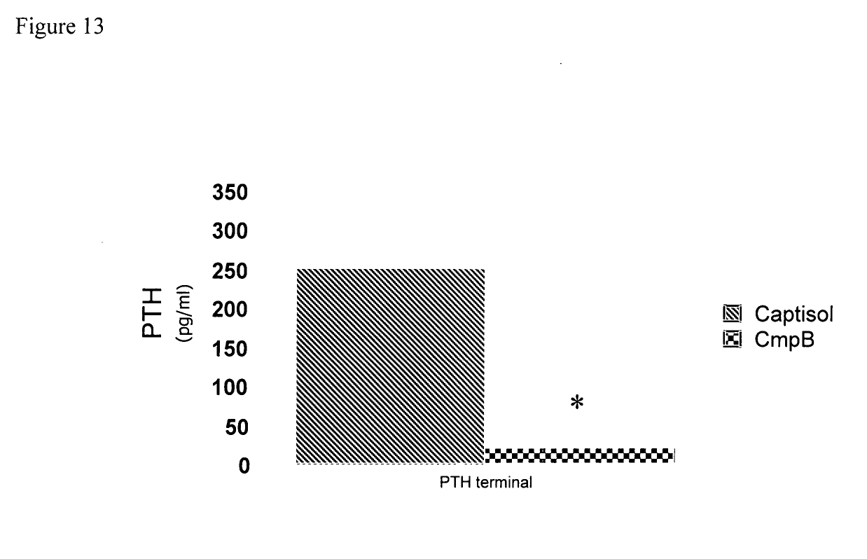

FIG. 13 demonstrates that administration of Compound B significantly reduced serum PTH levels (p<0.0001; ANOVA/Fisher's protected least squares differences post-hoc) when compared to vehicle treated animals.

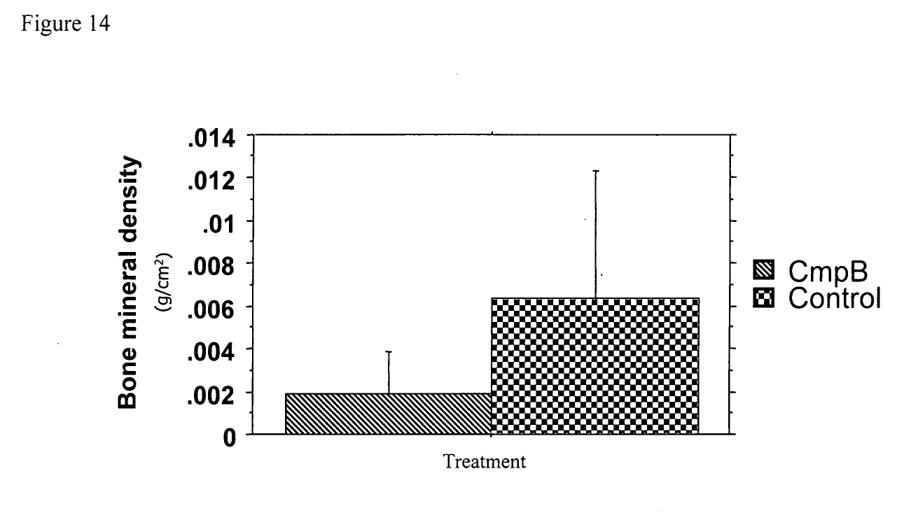

FIG. 14 demonstrates that administration of Compound B animals fed the adenine diet had a 75% reduction aortic bone mineral density, compared to vehicle treated animals on the same diet.

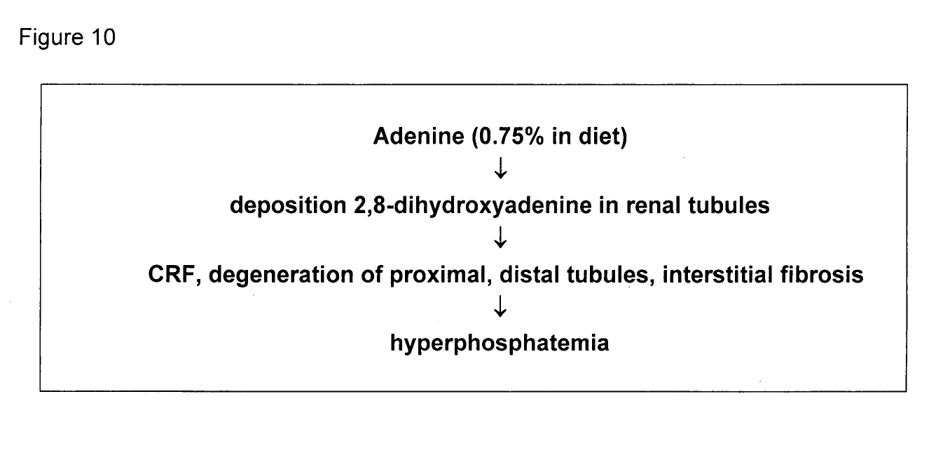

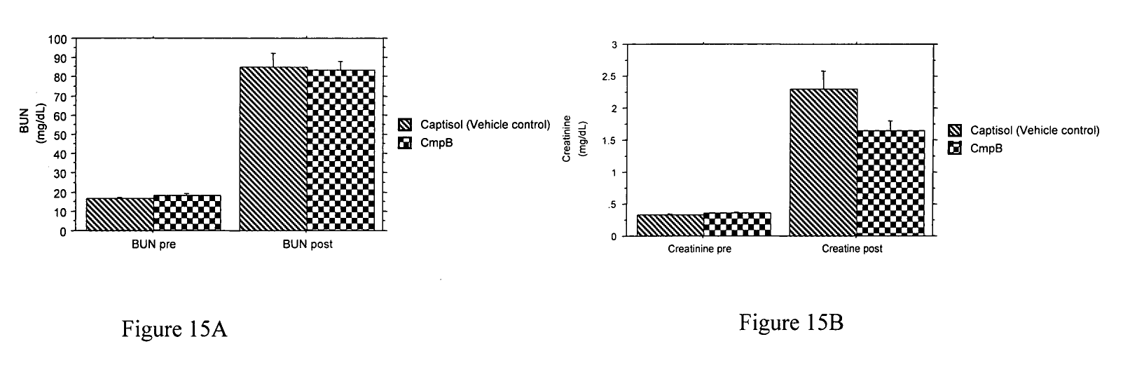

FIG. 15 illustrates the effect of Compound B on blood urea nitrogen (BUN) and creatinine in the adenine-induced CKD model with vascular calcification. Briefly, levels of both BUN and creatinine significantly (p<0.05) increased after 3 weeks adenine treatment (post) compared to pretreatment vehicle (n=4); Compound B (n=7) FIG. 10. There was no significant treatment effect of Compound B (n=7) on BUN (p>0.05) when compared to vehicle treated controls (n=4). However, Compound B (n=7) mediated a decrease in creatinine levels (p<0.05) compared to vehicle (n=4) treated animals.

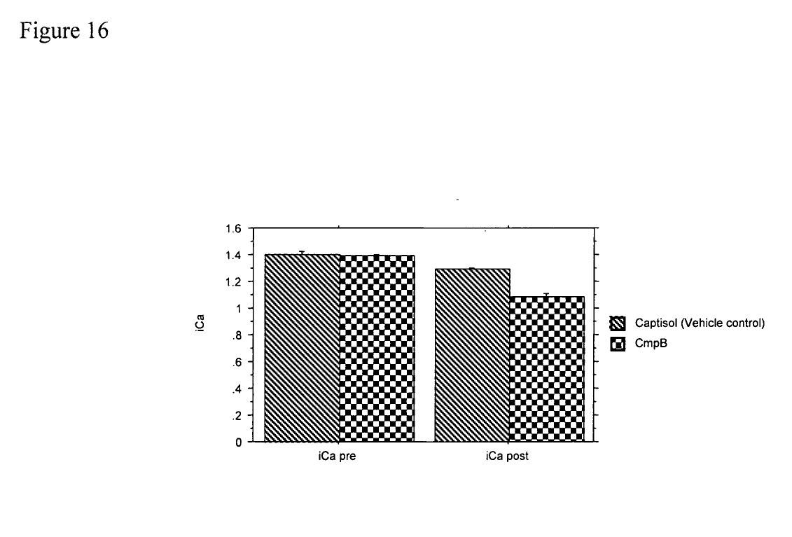

FIG. 16 demonstrates the effect of Compound B on ionized Ca in the adenine-induced CKD with vascular calcification. Serum ionized calcium (iCa) significantly decreased after 3 weeks adenine treatment (post) compared to pretreatment (pre) p<0.05; ANOVA; vehicle (n=4 pre; n=3 post); Compound B (n=8 pre; n=6 post). Compound B (n=6) treatment significantly reduced serum iCa compared to vehicle treatment (p=0.004; n=3).

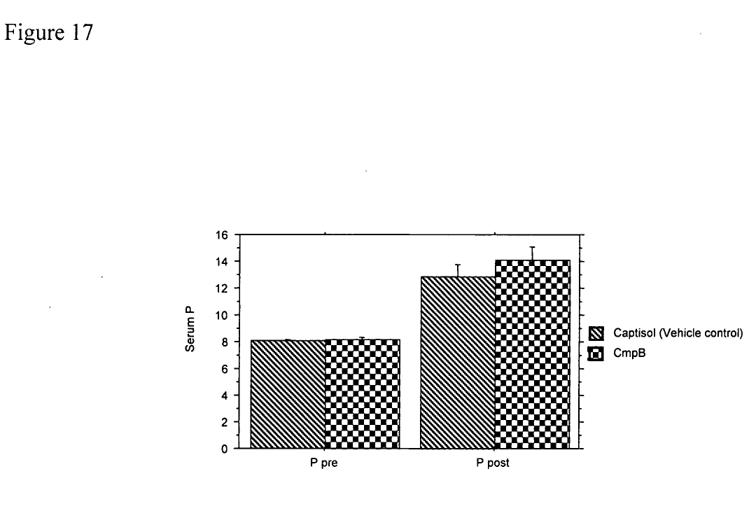

FIG. 17 demonstrates the effect of Compound B on serum phosphorus in the adenine-induced CKD with vascular calcification. Serum Phosphorous (P) significantly increased after 3 weeks adenine treatment (post) compared to pretreatment (pre) p>0.05; ANOVA; vehicle (n=4); Compound B (n=7). There was no significant (p>0.05) treatment effect of Compound B on serum P levels when compared to vehicle treated animals.

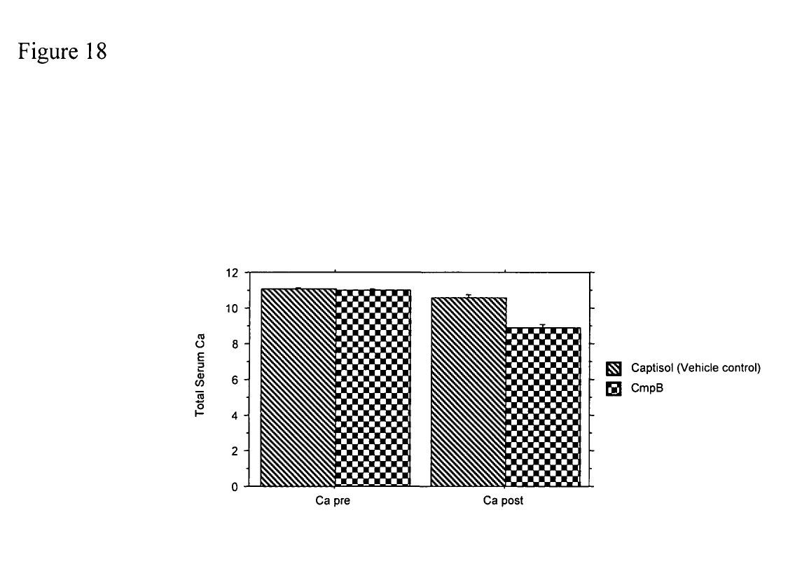

FIG. 18 demonstrates the effect of Compound B on serum Ca in the adenine-induced CKD with vascular calcification. Serum calcium (Ca) significantly decreased after 3 weeks adenine treatment (post) compared to pretreatment (pre) p<0.05; ANOVA; vehicle (n=4); Compound B (n=7). Compound B treatment significantly reduced total serum calcium compared to vehicle treatment (p=0.0001) ANOVA; vehicle (n=4); Compound B (n=7).

### EXAMPLE 3

This example demonstrates that the calcimimetic N-((6-(methyloxy)-4′-(trifluoromethyl)-1,1′-biphenyl-3-yl)methyl)-1-phenylethanamine (Compound B) significantly reduces paricalcitol-mediated increases in Ca and P content of aortic tissue from uremic animals.

Animals

Wistar rats (200-250g), fed a 0.6% Ca and 1.2% P diet, were 5/6 nephrectomized (5/6 Nx) or sham-operated (sham). Rats received the following treatments starting one day postsurgery: Sham+vehicle, 5/6 Nx+vehicle, 5/6 Nx+paricalcitol 240 ng/kg every 48 hr interperitoneally, 5/6 Nx+paricalcitrol+Compound B (1.5 mg/kg every 48 hr subcutaneously) or 5/6 Nx+Compound B (1.5 mg/kg every 48 hr subcutaneously). After 14 days, rats were anesthetized and sacrificed. The thoracic aorta was removed and processed for measurement of Ca and P content. Blood was collected at sacrifice to measure serum PTH, ionized Ca, P and creatinine. Results are summarized in Table 1 below.

TABLE 1

| | | Creatinine | Ionized Ca | | | Aortic Ca, | Aortic P, |
|---|---|---|---|---|---|---|---|
| | Treatment | mg/dL | mM | P mg/dL | PTH pg/ml | mg/g | mg/g |
| Sham | Vehicle | 0.56 ± 0.01 | 1.18 ± 0.02 | 6.3 ± 0.6 | 51.6 ± 18.4 | 2.1 ± 0.2 | 0.42 ± 0.17 |
| 5/6Nx | Vehicle | 1.13 ± 0.06 | 1.00 ± 0.04 | 13.7 ± 1.7 | 455 ± 70 | 3.3 ± 0.26 | 0.46 ± 0.11 |
| 5/6Nx | Paricalcitol | 2.32 ± 0.43 | Group | 14.7 ± 2.1 | 250 ± 61 | 9.5 ± 2.2 | 6.2 ± 2.4 |
| 5/6Nx | Comp B | 0.98 ± 0.1 | 0.93 ± 0.06 | 9.5 ± 1.5 | 45 ± 1.8 | 2.6 ± 0.16 | 0.33 ± 0.05 |
| 5/6Nx | Paricalcitol + | 1.14 ± 0.1 | 0.96 ± 0.03 | 9.9 ± 1.1 | 2.1 ± 0.6 | 3.6 ± 1.2 | 0.9 ± 0.9 |
| | Comp B | | | | | | |

5/6 Nx + vehicle

The procedure of 5/6 Nx mediated an increase in serum creatinine (p<0.05) that is consistent with chronic renal insufficiency. The significant decrease in serum ionized Ca (p<0.05), and significant increases in serum P and PTH (p<0.05) observed in the 5/6 Nx vehicle animals versus sham vehicle animals are all hallmarks of secondary hyperparathyroidism.

5/6 Nx+Paricalcitol

Administration of paricalcitol to 5/6 Nx rats significantly (p<0.05) decreased serum PTH levels compared to 5/6 Nx+vehicle. There were no changes in blood ionized calcium, creatinine or serum P levels compared to 5/6 Nx+vehicle. Paricalcitol significantly increased aortic Ca and P content (p<0.05) compared to vehicle treated 5/6 Nx animals (Table 1).

5/6 Nx+Compound B

Administration of Compound B to 5/6 Nx rats significantly (p<0.05) decreased serum PTH levels compared to vehicle treated 5/6 Nx animals. There were no changes in blood ionized calcium, creatinine or serum P levels compared to 5/6 Nx+vehicle. Likewise, administration of Compound B did not significantly change aortic Ca or P content when compared to either vehicle treated sham or 5/6 Nx animals (Table 1).

5/6 Nx+Paricalcitol+Compound B

Administration of Compound B to paricalcitol treated 5/6 Nx rats significantly (p<0.05) reduced aortic Ca and P content compared to 5/6 Nx rats treated with paricalcitol. The decrease in aortic Ca and P content by Compound B administration was not significantly different from sham controls treated with vehicle (normal levels). The combination of paricalcitol and Compound B further reduced serum PTH levels significantly (p<0.05) when compared to paracalcitrol or Compound B-treated 5/6 Nx rats alone. Animals treated with the combination of Compound B and paricalcitol showed no changes in blood ionized calcium, creatinine or serum P levels compared to 5/6 Nx+vehicle (Table 1).

### EXAMPLE 4

This example demonstrates that the calcimimetic N-((6-(methyloxy)-4′-(trifluoromethyl)-1,1′-biphenyl-3-yl)methyl)-1-phenylethanamine (Compound B) significantly reduces paricalcitol and calcitriol mediated mineralization of soft tissue.

Animals

Wistar rats (200-250g), fed a 0.6% Ca and 1.2% P diet, were 5/6 nephrectomized (5/6 Nx). Rats received the following treatments starting one day postsurgery: 5/6 Nx+vehicle, 5/6 Nx+paricalcitol 240 ng/kg every 48 hr or calcitriol 80 ng/kg every 48 hr interperitoneally, 5/6 Nx+paricalcitrol or calcitriol+Compound B (1.5 mg/kg every 48 hr subcutaneously) or 5/6 Nx+Compound B (1.5 mg/kg every 48 hr subcutaneously). After 14 days, rats were anesthetized and sacrificed and tissues were removed and processed for histological examination (Von Kossa staining to measure mineralization and H&E staining).

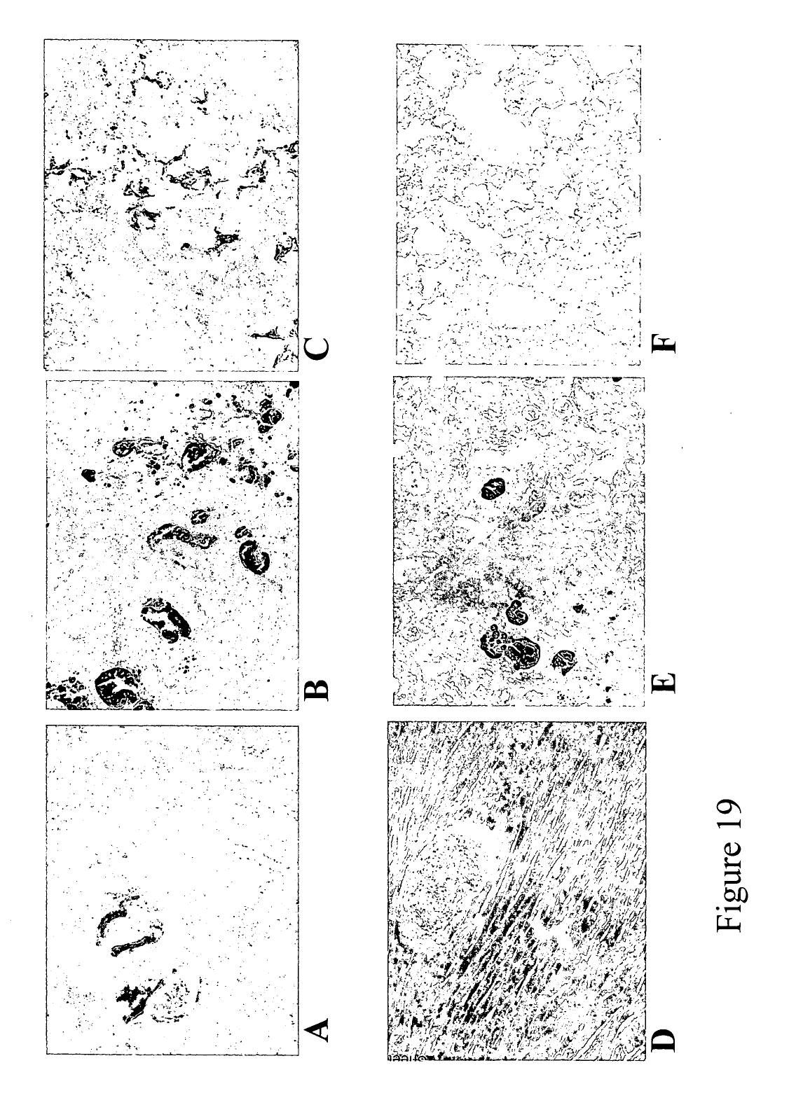

FIG. 19 (top panel: A, B, C) illustrates that administration of calcitriol to 5/6 Nx rats increased mineralization in heart (A), kidney (B), and lung (C), the only tissues examined, as evident by the dark tissue staining.

FIG. 19 (bottom panel: D, E, F) demonstrates that administration of Compound B to calcitriol-treated 5/6 Nx rats reduced heart (D), kidney (E), and lung (F) mineralization as shown by the reduced dark staining of tissues.

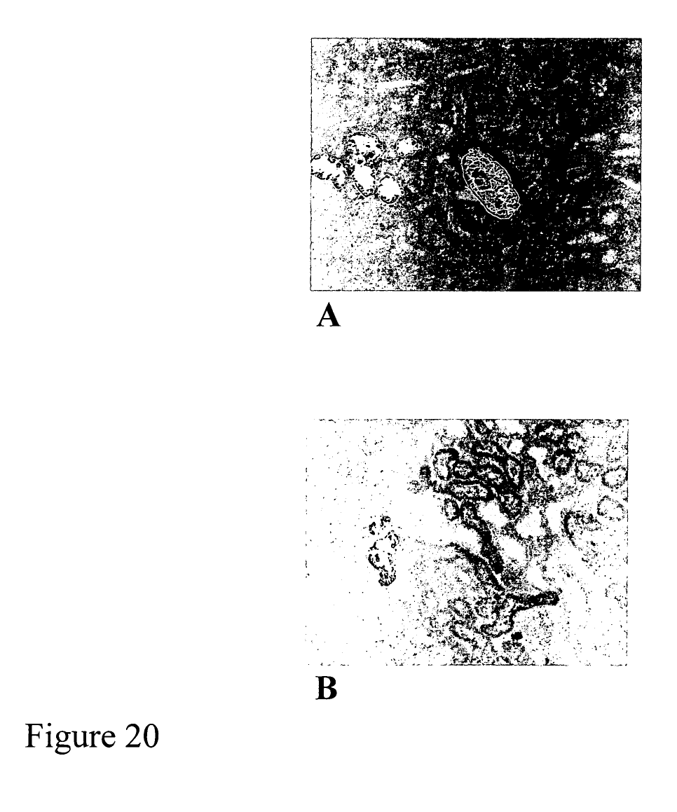

FIG. 20 (top panel: A) illustrates that administration of paricalcitol to 5/6 Nx rats increased mineralization in kidney (A), as evident by the dark tissue staining.

FIG. 20 (bottom panel: B) demonstrates that administration of Compound B to paricalcitol-treated 5/6 Nx rats reduced kidney (B) mineralization as evidenced by the reduced dark staining of tissues.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of treating vascular calcification in a subject comprising administering a therapeutically effective amount of a calcimimetic compound to the subject.

2. The method of claim 1, wherein the vascular calcification is atherosclerotic calcification.

3. The method of claim 1, wherein the vascular calcification is medial calcification.

4. The method of claim 1, wherein the subject is suffering from chronic renal insufficiency.

5. The method of claim 1, wherein the subject is suffering from end-stage renal disease.

6. The method of claim 1, wherein the subject is pre-dialysis.

7. The method of claim 1, wherein the subject is suffering from uremia.

8. The method of claim 1, wherein the subject is suffering from diabetes mellitus I or II.

9. The method of claim 1, wherein the subject is suffering from a cardiovascular disorder.

10. The method of any of claims 1-9, wherein the subject is human.

11. The method of any of claims 1-9, wherein the calcimimetic compound is a compound of the formula I 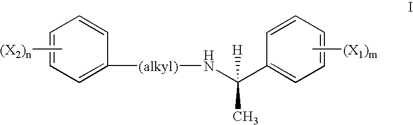
wherein:
X₁ and X₂, which may be identical or different, are each a radical chosen from CH₃, CH₃O, CH₃CH₂O, Br, Cl, F, CF₃, CHF₂, CH₂F, CF₃O, CH₃S, OH, CH₂OH, CONH₂, CN, NO₂, CH₃CH₂, propyl, isopropyl, butyl, isobutyl, t-butyl, acetoxy, and acetyl radicals, or two of X₁ may together form an entity chosen from fused cycloaliphatic rings, fused aromatic rings, and a methylene dioxy radical, or two of X₂ may together form an entity chosen from fused cycloaliphatic rings, fused aromatic rings, and a methylene dioxy radical; provided that X₂ is not a 3-t-butyl radical;
n ranges from 0 to 5;
m ranges from 1 to 5; and
the alkyl radical is chosen from C₁-C₃ alkyl radicals, which are optionally substituted with at least one group chosen from saturated and unsaturated, linear, branched, and cyclic C₁-C₉ alkyl groups, dihydroindolyl and thiodihydroindolyl groups, and 2-, 3-, and 4-piperidinyl groups;
or a pharmaceutically acceptable salt thereof.

12. The method of any of claims 1-9, wherein the calcimimetic compound is N-(3-[2-chlorophenyl]-propyl)-R-α-methyl-3-methoxybenzylamine or a pharmaceutically acceptable salt thereof.

13. The method of any of claims 1-9, wherein the calcimimetic compound is a compound of the formula II 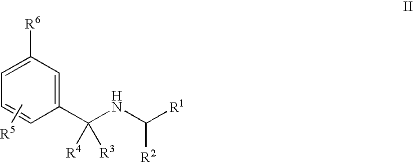
wherein:
R¹ is aryl, substituted aryl, heterocyclyl, substituted heterocyclyl, cycloalkyl, or substituted cycloalkyl;
R² is alkyl or haloalkyl;
R³ is H, alkyl, or haloalkyl;
R⁴ is H, alkyl, or haloalkyl;
each R⁵ present is independently selected from the group consisting of alkyl, substituted alkyl, alkoxy, substituted alkoxy, halogen, —C(═O)OH, —CN, —NR^{d}S(═O)ₘR^{d}, —NR^{d}C(═O)NR^{d}R^{d}, —NR^{d}S(═O)ₘNR^{d}R^{d} or —NR^{d}C(═O)R^{d};
R⁶ is aryl, substituted aryl, heterocyclyl, substituted heterocyclyl, cycloalkyl, or substituted cycloalkyl;
each R^{a} is, independently, H, alkyl or haloalkyl;
each R^{b} is, independently, aryl, aralkyl, heterocyclyl, or heterocyclylalkyl, each of which may be unsubstituted or substituted by up to 3 substituents selected from the group consisting of alkyl, halogen, haloalkyl, alkoxy, cyano, and nitro;
each R^{c} is, independently, alkyl, haloalkyl, phenyl or benzyl, each of which may be substituted or unsubstituted;
each R^{d} is, independently, H, alkyl, aryl, aralkyl, heterocyclyl, or heterocyclylalkyl wherein the alkyl, aryl, aralkyl, heterocyclyl, and heterocyclylalkyl are substituted by 0, 1, 2, 3 or 4 substituents selected from alkyl, halogen, haloalkyl, alkoxy, cyano, nitro, R^{b}, —C(═O)R^{c}, —OR^{b}, NR^{a}R^{a}, —NR^{a}R^{b}, —C(═O)OR^{c}, —C(═O)NR^{a}R^{a}, —OC(═O)R^{c}, —NR^{a}C(═O)R^{c}, —NR^{a}S(═O)ₙR^{c}and —S(═O)ₙNR^{a}R^{a};
m is 1 or 2;
n is 0, 1 or 2; and
p is 0, 1, 2, 3, or 4;
provided that if R² is methyl, p is 0, and R⁶ is unsubstituted phenyl, then R¹ is not 2,4-dihalophenyl, 2,4-dimethylphenyl, 2,4-diethylphenyl, 2,4,6-trihalophenyl, or 2,3,4-trihalophenyl;
or a pharmaceutically acceptable salt thereof.

14. The method any of claims 1-9, wherein the calcimimetic compound is N-((6-(methyloxy)-4′-(trifluoromethyl)-1,1′-biphenyl-3-yl)methyl)-1-phenylethanamine, or a pharmaceutically acceptable salt thereof.

15. The method of any of claims 1-9, wherein the calcimimetic compound is cinacalcet HCl.

16. The method of claim 1, wherein a vitamin D sterol had been previously administered to the subject.

17. The method of claim 16, wherein the vitamin D sterol is calcitriol, alfacalcidol, doxercalciferol, maxacalcitol or paricalcitol.

18. The method of claim 16, wherein the vitamin D sterol is calcitriol.

19. The method of claim 16, wherein the vitamin D sterol is paricalcitol.

20. The method of any of claims 1-9, wherein the calcimimetic compound is administered prior to or following administration of a vitamin D sterol.

21. The method of any of claims 1-9, wherein the calcimimetic compound is administered in combination with a vitamin D sterol.

22. The method of any of claims 1-9, wherein the calcimimetic compound is administered in combination with RENAGEL®.

23. A method of decreasing serum creatinine levels in a subject, comprising administering a therapeutically effective amount of a calcimimetic compound to the subject.

24. The method of claim 23, wherein the subject is suffering from increased serum creatinine levels induced by the administration of a vitamin D sterol to the subject.
